(19) United States
(12) Patent Application Publication
(10) Pub. No.: US 2020/0290035 A1
(43) Pub. Date: Sep. 17, 2020

(54) POINT-OF-CARE TESTING SYSTEM FOR BLOOD GASES AND CO-OXIMETRY

(71) Applicant: Relay Medical Corp, Toronto (CA)

(72) Inventor: James Samsoondar, Markham (CA)

(21) Appl. No.: 16/083,970

(22) PCT Filed: Mar. 27, 2017
(86) PCT No.: PCT/CA2017/050379

§ 371 Date: Sep. 11, 2018

Related U.S. Application Data
(60) Provisional application No. 62/316,653, filed on Apr. 1, 2016.

(51) Int. Cl. B01L 3/00; A61B 5/15

(52) U.S. Cl. CPC B01L 3/502; A61B 5/150022; A61B 5/150343; B01L 2300/0636; B01L 2300/0663; B01L 2300/1805; B01L 2400/0487; B01L 2200/025; B01L 2200/087; B01L 2400/0406; B01L 2200/148; B01L 2300/0861

(57) ABSTRACT

A cartridge and system involving a joint spectroscopic and biosensor blood analyzer for measurement of at least two hemoglobin species in a patient's blood sample by spectroscopy, and measurement of at least pH of the blood sample by biosensor, for assessing the patient's oxygenation and acid-base status. The cartridge comprises a housing having a first housing member and a second housing member bonded together by a gasket. The housing comprises a cartridge inlet; a blood storage conduit; an optical chamber; a biosensor conduit; a waste receptacle; a vent; an air bladder, an air bladder exit port; and, an optical window and an aligned optical member, the aligned optical member being one of a reflecting member or a second optical window, and being positioned to align with at least a portion of the optical chamber and at least a portion of the optical window.

### FIELD OF THE INVENTION

The invention relates to a disposable cartridge and an analyzer for point-of-care testing (POCT) of a patient's blood, using a combination of spectroscopic and biosensor measurements. In particular, the invention relates to POCT of blood gases and CO-oximetry.

### BACKGROUND OF THE INVENTION

There are many medical diagnostic tests that require a fluid, for example, blood (sometimes referred to as whole blood), serum, plasma, cerebrospinal fluid, synovial fluid, lymphatic fluid, calibration fluid, and urine. With respect to blood, a blood sample is typically withdrawn in either an evacuated tube containing a rubber septum, or a syringe, and sent to a central laboratory for testing. The eventual transfer of blood from the collection site to the testing site results in inevitable delays. Moreover, the red blood cells are alive and continue to consume oxygen during any delay in testing, which in turn changes the chemical composition of the blood sample, from the time the blood sample is collected to the time the blood sample is analyzed, also referred to as measured or tested.

One example of a blood analysis technique that is affected by delay in testing and transfer of blood from the blood collection device to the analyzer, is CO-oximetry. CO-oximetry is a spectroscopic technique that is used to measure the different Hemoglobin (Hb) species present in a blood sample, for example, Oxy-Hb, Deoxy-Hb, Met-Hb, Carboxy-Hb and Total-Hb. Some Co-oximeters can also measure Sulf-Hb and Fetal-Hb. The results of CO-oximetry are used to provide Hb Oxygen Saturation (sO₂) measurements in two ways: 1) functional sO₂ is defined as the ratio of Oxy-Hb to the sum of Oxy-Hb and Deoxy-Hb; and 2) fractional sO₂ is defined as the ratio of Oxy-Hb to the Total-Hb.

If the blood sample is exposed to air, the sO₂ measurements may become falsely elevated, as oxygen from the air is absorbed into the blood sample. CO-oximetry usually requires hemolyzing the red blood cells (hemolysis) using a sound generator, in order to make the blood sample more transparent for spectroscopic measurement; blood with intact red cells scatter significantly more electromagnetic radiation (EMR) than hemolyzed blood. Hemolysis can also be accomplished by mixing a chemical, for example, a detergent, with the blood. Parameters that can be measured in blood by spectroscopic techniques (or spectroscopy, sometimes referred to as spectrometry) are limited by the amount of EMR absorbed by the analytes measured. In contrast, for example, without limitation, hydrogen ions (which determine pH) and electrolytes (e.g., sodium, potassium, and chloride) do not absorb EMR in the approximate wavelength range of about 300 nm to 2500 nm. Therefore, if this wavelength range is used to conduct spectroscopic measurements of Hb species, then these important parameters, i.e., hydrogen ions and electrolytes, must be measured by another means.

Another example of a blood analysis technique that is affected by the aforementioned sources of error is blood gases. Traditionally, blood gas measurement includes the partial pressure of oxygen (pO₂), the partial pressure of carbon dioxide (pCO₂), and pH. From these measurements, other parameters can be calculated, for example, sO₂, bicarbonate, base excess and base deficit. Blood gas and electrolyte measurements usually employ biosensors, also referred to as electrochemical sensors or electrochemical detectors. Bench-top analyzers are available, which perform the following: (1) measurement of blood gases, (2) CO-oximetry, or (3) combined measurement of blood gases and CO-oximetry. Some combinations of diagnostic measurement instruments also include electrolytes, and other measurements, for example, lactate and creatinine. Because these instruments are large and expensive, they are usually located in central laboratories. Biosensor technology is also limited by the blood parameters biosensors can measure. To the inventor's knowledge, biosensors are not currently available for performing CO-oximeters. U.S. Pat. Nos. 5,096,669 and 7,094,330 to Lauks et al., as examples, describe in details cartridges that employ biosensor technology for POCT. In particular, they teach about pH measurement (a potentiometric measurement), blood gas measurement (a potentiometric and an amperometric measurement for pCO₂ and pO₂, respectively), and hematocrit measurement (a conductivity measurement). U.S. Pat. No. 7,740,804 to Samsoondar (the present inventor) teaches disposable cartridges for spectroscopic measurement (e.g., CO-oximetry) for POCT using unaltered blood. U.S. Pat. Nos. 5,430,542 and 6,262,798 to Shepherd describes a method for making disposable cuvettes having a path length in the range of 80 to 130 micrometers for performing CO-oximetry measurement on unaltered blood.

Blood tests for assessing a patient's oxygenation and acid-base status may include pH, sO₂, CO₂, and Total Hb. The leading POCT analyzers used to assess a patients acid-base status estimate sO₂ from a measured partial pO₂, and estimate Total Hb from a measured hematocrit. Both hematocrit and pO₂ are measured using biosensors.

sO₂ calculated from pO₂ is criticized in the literature because: 1) pO₂ measures the O₂ dissolved in the blood plasma, which accounts for only about 1% of the total oxygen in blood—the remaining 99% of blood oxygen is bound to Hb; 2) it is assumed that the patient's red blood cells (RBC) contain normal levels of 2,3-diphosphoglycerate; and 3) the patient has normal levels of dyshemoglobins, e.g., Carboxy-Hb and Met-Hb. Dyshemoglobins are non-functional Hbs. Temperature and pH, which are also sources of error, are usually corrected for.

Total Hb estimated from hematocrit measurement by conductivity is criticized in the literature because: 1) a certain RBC Hb concentration is assumed for all patients; and 2) alteration in plasma protein, electrolytes, white cells, and lipids are sources of errors in hematocrit measurement. These assumptions can lead to significant errors in managing seriously ill patients. Moreover, Hb measurement is preferred over hematocrit measurement for evaluating chronic anemia and blood loss. Unnecessary blood transfusion due to underestimation of Hb from hematocrit is a major concern.

In choosing a POCT analyzer, a user must understand clearly the parameters that are actually measured and the parameters that are calculated from measured parameters. Measurement of Total Hb and sO₂ performed by spectroscopy provide the best measurement of a patient's oxygenation status, because they are more accurate than results calculated from hematocrit and pO₂, respectively. Lab analyzers can easily combine biosensor and spectroscopic technologies because analyzer size is not a limitation. Currently, no small POCT analyzer is available that provides blood gases (includes pH) and CO-oximetry. Some POCT vendors provide a solution in the form of a separate POCT analyzer just for performing CO-oximetry, which complements their blood gas POCT analyzer.

Since CO-oximetry measures functional Hb species, and non-functional Hb species like Carboxy-Hb and Met-Hb, a physician can continue to confidently monitor a patient's oxygenation status non-invasively using a Pulse Oximeter. According to best practice, pulse oximetry should only be used after verifying that the patient's blood does not contain significant amount of non-functional Hb. The presence of elevated non-functional hemoglobin species is a source of error in pulse oximetry. The present invention can use capillary blood as well as arterial blood, which provides a major advantage for babies. Obtaining arterial blood is painful, can cause nerve damage, must be performed by a qualified person like a physician, and the resulting blood loss in babies is clinically significant. The cartridge of the present invention can also facilitate monitoring Met-Hb in neonates during treatment with nitric oxide for respiratory distress, and facilitate measuring bilirubin for assessing neonatal jaundice. The use of capillary blood also makes the present invention an attractive tool for monitoring sO₂, Carboxy-Hb (increased due to carbon monoxide poisoning resulting from smoke inhalation) and pH in firefighters and other victims of smoke inhalation. Most of these victims will be treated with oxygen, which elevates the pO₂, therefore pO₂ cannot be used to assess the blood oxygen content. CO-oximetry is therefore essential to victims of smoke inhalation. Capillary blood is usually obtained from a finger, heel or ear lobe prick. The capillary blood can be altered (“arterialized”) to more closely resemble arterial blood by applying a heating pad to the site that will be pricked.

U.S. Pat. No. 8,206,650 to Samsoondar (the present inventor) teaches the combination of spectroscopy and biosensor technologies in one disposable cartridge, and can therefore provide pH, blood gases and CO-oximetry on a small POCT analyzer. The users are provided with the convenience of applying the sample once, as opposed to using a first analyzer that employs biosensor technology alone, and a second analyzer that employs spectroscopy alone. However, U.S. Pat. No. 8,206,650 does not provide details required by a person with ordinary skill in the art, for making a functional cartridge, and further does not provide details that can be applied to a cartridge manufacturing process.

U.S. Pat. No. 8,206,650 provides a single cartridge option that can be used to test blood from a syringe like arterial blood, and capillary blood at the surface of a body part, which is a very important consideration when the patient is a neonate. However, the option for obtaining capillary blood is limited. A person of ordinary skill in the art of blood gases will appreciate that the pO₂ will be overestimated significantly due to atmospheric contamination; current practice includes inserting the open end of a capillary tube inside the drop of blood, quickly sealing the ends of the capillary tube, and taking the sample to an analyzer.

U.S. Pat. No. 9,470,673. to Samsoondar (the present inventor), the contents of which are hereby incorporated in entirety by reference, provides improvements to the teachings of U.S. Pat. No. 8,206,650, for example, the design of a capillary adaptor for drawing capillary blood into the cartridge, the design of a more efficient capillary break, the design of the delivery system for calibration fluid, and the design of the analyzer cartridge receptor. Other limitations of the cartridge described in U.S. Pat. No. 9,470,673 will become apparent as different embodiments of the present invention are described.

### SUMMARY OF THE INVENTION

In accordance with an aspect of an embodiment of the present invention there is provided a disposable cartridge for operation with a joint spectroscopic and biosensor blood analyzer for measurement of at least two hemoglobin species in a patient's blood sample by spectroscopy, and measurement of at least pH of the blood sample by biosensor, for assessing the patient's oxygenation and acid-base status. The cartridge comprises a housing having at least a first housing member and a second housing member bonded together by a gasket. The housing comprises a cartridge inlet; a blood storage conduit within the housing having a proximal end close to the cartridge inlet and a distal end away from the cartridge inlet; an optical chamber within the housing for receiving the blood from the distal end of the blood storage conduit and for measuring the at least two hemoglobin species; an optical chamber overflow chamber for receiving the blood from the optical chamber; a biosensor conduit within the housing for receiving the blood from the optical chamber overflow chamber, the biosensor conduit comprising a proximal end, a distal end and at least a portion of a pH biosensor; a waste receptacle for receiving liquid waste from the biosensor conduit; a vent for relieving pressure in the waste receptacle; an air bladder and an air bladder exit port within the housing for providing pressurized air for urging blood from the blood storage conduit into the biosensor conduit; and, an optical window and an aligned optical member, the first housing member comprising one of the optical window and the aligned optical member, and the second housing member comprising the other of the optical window and the aligned optical member; the aligned optical member being one of a reflecting member or a second optical window, and being positioned to align with at least a portion of the optical chamber and at least a portion of the optical window. The gasket has at least one gasket cut-out positioned to provide fluid connection between the blood storage conduit and the optical chamber, wherein at least a portion of the at least one gasket cut-out is positioned to align with at least a portion of the optical chamber for collecting spectroscopic data from blood in that portion of the optical chamber.

In some embodiments, the at least one gasket cut-out has a second portion positioned to align with the active area of the pH biosensor.

In some embodiments, the disposable cartridge is insertable into a receptor of the joint spectroscopic and biosensor analyzer, and at least one of the first and second optical window is positioned to align with at least a portion of the optical chamber for collecting spectroscopic data from blood in that portion of the optical chamber. In these embodiments, the housing further comprises a blood shunt for providing fluid connectivity between the distal end of the blood storage conduit and the optical chamber overflow chamber. The optical chamber overflow chamber comprises: a first duct fluidly connected with the blood shunt and traversing a thickness of the second housing member; a recess disposed at the bottom of the second housing member and fluidly connected to the first duct; and, a second duct having a first cross-sectional area, and fluidly connected to the recess. In addition to that blood shunt, the housing further comprises an enlarged cavity having a second cross-sectional area parallel to the first cross-sectional area; wherein, the second cross-sectional area is substantially larger than the first cross-sectional area, whereby blood flow by capillary action slows down as the blood reaches the end of the second duct, and wherein the enlarged cavity is simultaneously in fluid connection with the optical chamber and the second duct.

In some embodiments, this disposable cartridge is insertable along a plane substantially defined by a surface of the gasket, into the receptor of the joint spectroscopic and biosensor analyzer, the optical chamber comprising an optical depth dimension orthogonal to the plane, the blood shunt having a maximum shunt depth dimension orthogonal to the plane, the maximum shunt depth dimension being substantially larger than the optical chamber depth dimension, and the first cross-sectional area being along the place.

In some embodiments of this disposable cartridge, the optical chamber overflow chamber is fluidly connected with the optical chamber, and the housing further comprises: a calibration fluid pouch for storing and releasing calibration fluid; a spike disposed in the second housing member of the cartridge for rupturing the calibration fluid pouch; a recess disposed in the opposite side of the second housing member; and a hole in the spike for permitting flow of the calibration fluid from the calibration fluid pouch to the recess for channeling the calibration fluid to the biosensor conduit.

In some embodiments, this cartridge further comprises a compressible member surrounding the spike, for supporting the calibration fluid pouch.

In accordance with another aspect of another embodiment of the present invention, there is provided a system for transferring capillary blood from a puncture site of a body part of a patient, the system comprising a capillary adaptor; and a disposable cartridge according to any of the embodiments described above. The cartridge inlet further comprises: an internal wall for receiving the capillary adaptor, the internal wall defining an airflow path for airflow between an exterior of the disposable cartridge and the blood storage conduit when the capillary adaptor is being removed from the cartridge inlet; an external wall having a cartridge inlet thread; a blood storage conduit entrance at the base of the cartridge inlet, the blood storage conduit beginning at the blood storage conduit entrance; and a cartridge inlet inner face surrounding the blood storage conduit entrance. The capillary adaptor comprises a capillary adaptor inlet member comprising a capillary adaptor tube, the capillary adaptor inlet member having a capillary adaptor inlet port for receiving the blood sample; a capillary adaptor outlet member sized to fit into the cartridge inlet; a capillary adaptor outlet port disposed at the end of the capillary adaptor outlet member; a capillary adaptor face surrounding the capillary adaptor outlet port; a capillary adaptor lumen extending from the capillary adaptor inlet port to the capillary adaptor outlet port; a handgrip for handling the capillary adaptor; and an internal wall in the hand grip having a capillary adaptor thread for engaging the cartridge inlet thread in the cartridge inlet. When the capillary adaptor thread is properly engaged with the cartridge inlet thread, the capillary adaptor face mates with the cartridge inlet inner face, sufficiently to permit flow of blood from the patient to the blood storage conduit by capillary action.

In some embodiments of this system, the position of the capillary adaptor face relative to the cartridge inlet inner face, when the capillary adaptor is fully engaged with the cartridge inlet, is one of no gap between the capillary adaptor face.

In some embodiments of this system, the cartridge inlet thread is an abbreviated thread.

In some embodiments of this system, the capillary adaptor thread is an abbreviated thread.

In some embodiments of this system, the volume of the capillary adaptor lumen is in the approximate range of about 5 microliters to about 20 microliters.

In some embodiments of this system, the volume of the capillary adaptor lumen is in the approximate range of about 5 microliters to about 10 microliters.

In some embodiments of this system, the length of the capillary adaptor inlet member is in the approximate range of about 2 millimeters to about 5 millimeters.

In accordance with another aspect of an embodiment of the present invention, there is provided a system for transferring blood from a syringe containing the blood. The system comprises the disposable cartridge according to any of the embodiments described above, wherein the cartridge inlet engages the syringe. The cartridge inlet further comprises an internal wall for receiving the syringe, the internal wall defining an airflow path for airflow between an exterior of the disposable cartridge and the blood storage conduit when the syringe is being removed from the cartridge inlet; an external wall; a blood storage conduit entrance at the base of the cartridge inlet, the blood storage conduit beginning at the blood storage conduit entrance; and a cartridge inlet inner face surrounding the blood storage conduit entrance.

In accordance with yet another aspect of an embodiment of the present invention, there is provided a joint spectroscopic and biosensor system for measurement of at least two hemoglobin species in a patient's blood sample by spectroscopy, and measurement of at least pH of the blood sample by biosensor, for assessing the patient's oxygenation and acid-base status. The system comprises the disposable cartridge according to any of the embodiments described above, wherein the first optical window and the second optical window are part of the optical chamber; the optical chamber overflow chamber is fluidly connected with the optical chamber, and the housing further comprises: a pH biosensor electrical output element; and a calibration fluid pouch containing calibration fluid for at least calibrating the pH biosensor; and an analyzer comprising an analyzer housing. The analyzer housing comprises: a receptor comprising a first opening for receiving and aligning the cartridge in an operational position; a source of electromagnetic radiation; at least one photodetector; a power supply; and a processor for controlling the analyzer. The receptor further comprises: a second opening for directing the electromagnetic radiation to the first optical window when the cartridge is in the operational position; a third opening for directing electromagnetic radiation emerging from the second optical window to the at least one photodetector when the cartridge is in the operational position; a physical interface for providing electrical contact between the pH biosensor electrical output element and the processor; and a bracket mounted on the receptor for at least supporting a first stepper motor for applying force to the calibration fluid pouch against a spike for rupturing the calibration fluid pouch to release the calibration fluid, and a second stepper motor for forcing air from the air bladder through the air bladder exit port, for pushing the blood into the biosensor conduit.

In some embodiments of this system, the receptor further comprises a top portion and a bottom portion, and the bottom portion comprises at least one heating element layered on a surface of the bottom portion, for heating the cartridge, and the top portion comprises a spring-loaded locating element for engaging with a notch disposed at a top of the cartridge, for forcing the cartridge against the at least one heating element.

In some embodiments of the system, whether comprising a capillary adapter or for use with the syringe, the system further comprises a cap for covering the cartridge inlet when one of the capillary adaptor and the syringe is withdrawn from the cartridge inlet. The cap comprises a cap airflow path for airflow between an exterior of the disposable cartridge and the blood storage conduit when the cap is being engaged to impede blood in the blood storage conduit being disturbed by compression of air within the cartridge inlet during engagement of the cap.

Other aspects and features of the present invention will become apparent to those having ordinary skill in the art, upon review of the following description of the specific embodiments of the invention.

### BRIEF DESCRIPTION OF THE DRAWINGS

For a better understanding of the present invention, and to show more clearly how it may be carried into effect, reference will now be made, by way of example, to the accompanying drawings, which illustrate aspects of embodiments of the present invention and in which:

FIG. 1A is an exploded view of a first embodiment of a joint-diagnostic spectroscopic and biosensor cartridge 10 for use with a joint-diagnostic spectroscopic and biosensor analyzer;

FIG. 1B is a top view of the second housing member 30 of the cartridge, with the biosensor array 80 installed in the receptacle 83, shown in FIG. 1A;

FIG. 1C is a bottom view of the first housing member 20 of the cartridge shown in FIG. 1A;

FIG. 1D is a top view of gasket 100 of the cartridge shown in FIG. 1A;

FIG. 1E is the top view of the second housing member 30 shown in FIG. 1B, overlaid by and in alignment with the gasket 100 shown in FIG. 1D,

FIG. 1F is the bottom view of the first housing member 20 shown in FIG. 1C, overlaid by and in alignment with the gasket 100 shown in FIG. 1D,

FIG. 2A is a perspective view of the joint-diagnostic spectroscopic and biosensor cartridge 10 shown in FIG. 1A;

FIG. 2B is a first perspective view of an embodiment of a capillary adaptor 70 for use with cartridge 10 shown in FIG. 2A;

FIG. 2C is a second perspective view of the capillary adaptor 70 shown in FIG. 2B;

FIG. 2D is the capillary adaptor 70 shown in FIG. 2B, engaged with the inlet 43 of the cartridge 10 shown in FIG. 2A;

FIG. 2E is a detailed view of the detail E of the cartridge 10 shown in FIG. 2A;

FIG. 2F is a first perspective view of an embodiment of a cap 60 for use with cartridge 10 shown in FIG. 2A;

FIG. 2G is a second perspective view of the cap 60 shown in FIG. 2F;

FIG. 2H is the cap 60 shown in FIG. 2F, engaged with the inlet 43 of the cartridge 10 shown in FIG. 2A;

FIG. 3A is a top view of the cartridge and capillary adaptor shown in FIG. 2D;

FIG. 3B is a first cross-sectional view through the cartridge and capillary adaptor shown in FIG. 3A along line B-B;

FIG. 3C is a front view of the cartridge and capillary adaptor shown in FIG. 3A;

FIG. 3D is a second cross-sectional view through the cartridge and capillary adaptor shown in FIG. 3A along line D-D;

FIG. 3E is a detailed view of the detail E of the cartridge and capillary adaptor shown in FIG. 3D;

FIG. 3F is a detailed view of the detail F of the cartridge and capillary adaptor shown in FIG. 3B;

FIG. 4A is a top view of the cap 60 shown in FIG. 2F;

FIG. 4B is a right side view of the cap shown in FIG. 4A;

FIG. 4C is a bottom view of the cap shown in FIG. 4A;

FIG. 4D is a top view of the capillary adaptor 70 shown in FIG. 2B;

FIG. 4E is a right side view of the capillary adaptor shown in FIG. 4D;

FIG. 4F is a bottom view of the capillary adaptor shown in FIG. 4D;

FIG. 4G is a first perspective view of the capillary adaptor shown in FIG. 4D;

FIG. 4H is a second perspective view of the capillary adaptor shown in FIG. 4D;

FIG. 5A is a top view of the cartridge shown in FIG. 1A;

FIG. 5B is a first cross-sectional view through the cartridge shown in FIG. 5A along line B-B;

FIG. 5C is a detailed view of the detail C of the cartridge shown in FIG. 5B,

FIG. 5D is a second cross-sectional view through the cartridge shown in FIG. 5A along line D-D;

FIG. 5E is a detailed view of the detail E of the cartridge shown in FIG. 5A;

FIG. 5F is a detailed view of the detail F of the cartridge shown in FIG. 5D;

FIG. 5G is a third cross-sectional view through the cartridge shown in FIG. 5A along line G-G;

FIG. 5H is a detailed view of the detail H of the cartridge shown in FIG. 5G.

FIG. 6A is an exploded view of a second embodiment of a joint-diagnostic spectroscopic and biosensor cartridge 10a for use with a joint-diagnostic spectroscopic and biosensor analyzer;

FIG. 6B is a top view of the second housing member 30a of the cartridge, with the biosensor array 80a installed in the receptacle 83a, shown in FIG. 6A;

FIG. 6C is a bottom view of the first housing member 20a of the cartridge shown in FIG. 6A;

FIG. 6D is a top view of gasket 100a of the cartridge shown in FIG. 6A;

FIG. 6E is the top view of the second housing member 30a shown in FIG. 6B, overlaid by and in alignment with the gasket 100a shown in FIG. 6D;

FIG. 6F is the bottom view of the first housing member 20a shown in FIG. 6C, overlaid by and in alignment with the gasket 100a shown in FIG. 6D;

FIG. 7A is a perspective view of the joint-diagnostic spectroscopic and biosensor cartridge 10a shown in FIG. 6A, with the first housing member 20a exposed and a capillary adaptor 70a engaged with the inlet 43a;

FIG. 7B is a second perspective view of cartridge 10a shown in FIG. 7A, with air bladder cavity 86a and calibration fluid pouch 90a exposed by hiding perforated label 170, and a cap 60a engaged with the inlet 43a;

FIG. 7C is a third perspective view of the joint-diagnostic spectroscopic and biosensor cartridge 10a shown in FIG. 7A, with the second housing member 30a exposed;

FIG. 7D is a fourth perspective view of cartridge 10a shown in FIG. 7A, with recesses 147 and 149 in the bottom of second housing member 30a exposed by hiding bottom cover 150;

FIG. 7E is a top view of cartridge 10a, with cap 60a partly engaged with the inlet 43a;

FIG. 7F is a cross-sectional view through the cartridge and cap shown in FIG. 7E along line F-F;

FIG. 7G is a detailed view of the detail G of the cartridge and cap shown in FIG. 7F;

FIG. 8A is a top view of the cartridge 10a shown in FIG. 6A;

FIG. 8B is a first cross-sectional view through the cartridge shown in FIG. 8A along line B-B;

FIG. 8C is a second cross-sectional view through the cartridge shown in FIG. 8A along line C-C,

FIG. 8D is a detailed view of the detail D of the cartridge shown in FIG. 8C,

FIG. 8E is a detailed view of the detail E of the cartridge shown in FIG. 8B;

FIG. 8F is a third cross-sectional view through the cartridge shown in FIG. 8A along line F-F;

FIG. 8G is a fourth cross-sectional view through the cartridge shown in FIG. 8A along line G-G;

FIG. 8H is a fifth cross-sectional view through the cartridge shown in FIG. 8A along line H-H;

FIG. 8J is a detailed view of the detail J of the cartridge shown in FIG. 8F;

FIG. 8K is a detailed view of the detail K of the cartridge shown in FIG. 8G;

FIG. 8L is a detailed view of the detail L of the cartridge shown in FIG. 8H;

FIG. 9A is a perspective view of a joint-diagnostic spectroscopic and biosensor cartridge inserted in the receptor of an analyzer;

FIG. 9B is a top view of a joint-diagnostic spectroscopic and biosensor cartridge inserted in the receptor of an analyzer;

FIG. 9C is a first cross-sectional view through the cartridge and receptor shown in FIG. 9B along line C-C,

FIG. 9D is a second cross-sectional view through the cartridge and receptor shown in FIG. 9B along line D-D;

FIG. 9E is a third cross-sectional view through the cartridge and receptor shown in FIG. 9B along line E-E;

FIG. 9F is a second perspective view a joint-diagnostic spectroscopic and biosensor cartridge inserted in the receptor of an analyzer, with the top portion of the receptor 220 hidden;

FIG. 9G is a perspective of the cartridge 10a shown in FIG. 9F;

FIG. 9H is a second perspective of the cartridge 10a, showing the second housing member 30a;

FIG. 9J is a perspective view of the bottom portion 230 of the cartridge receptor 200 shown in FIG. 9A, with the top portion of the cartridge receptor 220 and cartridge 10a hidden; and

FIG. 10 is a block diagram of an embodiment of a joint-diagnostic spectroscopic and biosensor analyzer.

### DETAILED DESCRIPTION OF PREFERRED ASPECTS OF THE INVENTION

The invention provides a system for joint spectroscopic and biosensor measurement of at least two hemoglobin species in a patient's blood sample by spectroscopy, and measurement of at least the blood pH by biosensor. The terms biosensor, electrochemical sensor and electrochemical detector are sometimes used interchangeably, and they have the same meaning in this description. The system comprises a disposable cartridge adapted for insertion into a receptor of an analyzer, a cap for capping the cartridge when it is inserted into the analyzer, and a capillary adaptor for drawing blood directly from the puncture site of the skin of a patient, into the cartridge. The results are used for assessing a patient's oxygenation and acid-base status. This system allows for the use of capillary blood instead of arterial blood, which is particularly useful for neonatal care.

Some embodiments of an analysis system include at least some of the following: an analyzer described in part in U.S. Pat. Nos. 8,206,650 and 9,470,673, the analyzer having some of the following: i) a power supply, which is optionally in the form of disposable or rechargeable batteries; ii) a source of electromagnetic radiation (EMR), for example, one or more LEDs, a tungsten lamp, one or more lasers, or any combination thereof; iii) a receptor in the analyzer housing for receiving a disposable cartridge; iv) a photodetector for measuring EMR transmitted through or reflected from a blood sample within the optical chamber of the cartridge and for providing an EMR-based signal derived from the EMR transmitted through or reflected from the blood sample; v) a processor for controlling the analyzer and in communication with the photodetector for receiving the EMR-based signal, and at least one calibration algorithm installed in the processor for transforming the EMR-based signal into a hemoglobin specie concentration; vi) a physical interface attached to the receptor for connecting with an analyzer processor and for connecting with the biosensor; vii) means for releasing the calibration fluid from the calibration fluid pouch and transporting released calibration fluid to the biosensor conduit for calibrating at least the pH biosensor prior to measuring the pH of the blood sample; viii) means for maintaining the active area of the biosensor at a pre-determined temperature; and ix) means for preheating the blood sample.

When the biosensor electrical contact of the cartridge is inserted into the physical interface of the receptor, the optical chamber of the cartridge becomes positioned to receive the EMR from the EMR source.

Some embodiments of the system also include: x) means for handling the blood sample, for example, a) a syringe containing the blood, and b) a capillary adaptor capable for transferring capillary blood directly from punctured skin of the body part of a patient to the cartridge; and xi) a cap for sealing the cartridge inlet. A syringe is required for collecting arterial blood, but capillary blood can be obtained by puncturing a body part with a lancet, and transferring the capillary blood that accumulates at the surface of the skin, to the cartridge via a capillary adaptor. In certain situations, for example when the patient is a baby, and under certain conditions, capillary blood may be used as a substitute for arterial blood. Moreover, collection arterial blood is painful, may cause nerve damage, and is usually performed by a physician.

The means for calibrating the at least one biosensor includes: a) a pouch within the housing containing calibration fluid; b) means for releasing fluid from the calibration pouch; and c) a calibration fluid conduit for transporting the released calibration fluid to the biosensor conduit. Those skilled in the art will appreciate that the electrical signals generated from the biosensor after it comes in contact with a calibration fluid of known composition, and the known concentration of the analyte in the calibration fluid, can be used to generate a calibration algorithm for the analyte, and therefore for the sake of brevity, the mathematics involved in biosensor calibration will not be discussed here. Some embodiments of the present invention provide biosensor calibration algorithms installed in the analyzer processor, and therefore do not require the calibration fluid.

The current practice when testing capillary blood on a blood gas analyzer or a CO-oximeter is to collect the capillary blood in a capillary tube, and subsequently transfer the blood from the capillary tube to the analyzer. This transfer of the blood from the capillary tube to the analyzer presents sources of error, for example: a) cellular metabolism continues after blood is collected, and the error is proportional to the delay in testing; and b) opportunity for atmospheric contamination by incorporation of air bubbles in the capillary tube, which is subsequently mixed into the blood. An external magnet is used to move a piece of wire located inside the capillary tube, forward and backward along the capillary tube, in order to mix the sample with anticoagulant deposited on the internal wall of the capillary tube. The present invention provides a capillary adaptor designed to eliminate this step of sample transfer. The atmosphere contains about 21% oxygen; therefore for direct measurement (CO-oximetry) or indirect measurement (i.e., calculating sO₂ from measured pO₂) of oxygen saturation, the blood must be protected from atmospheric contamination in order to minimize errors, and delay in testing must be minimized.

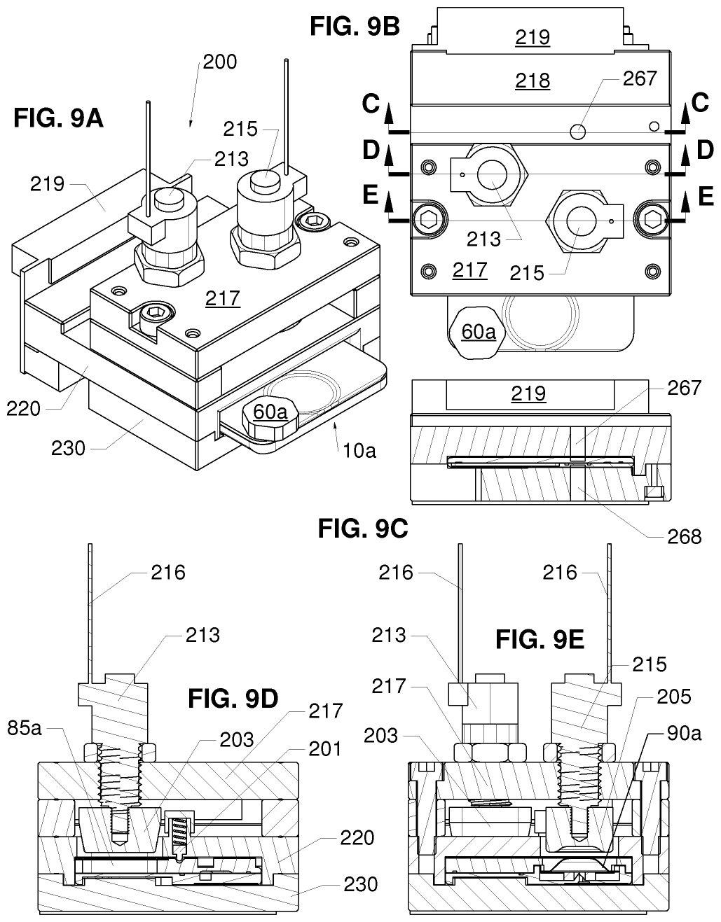

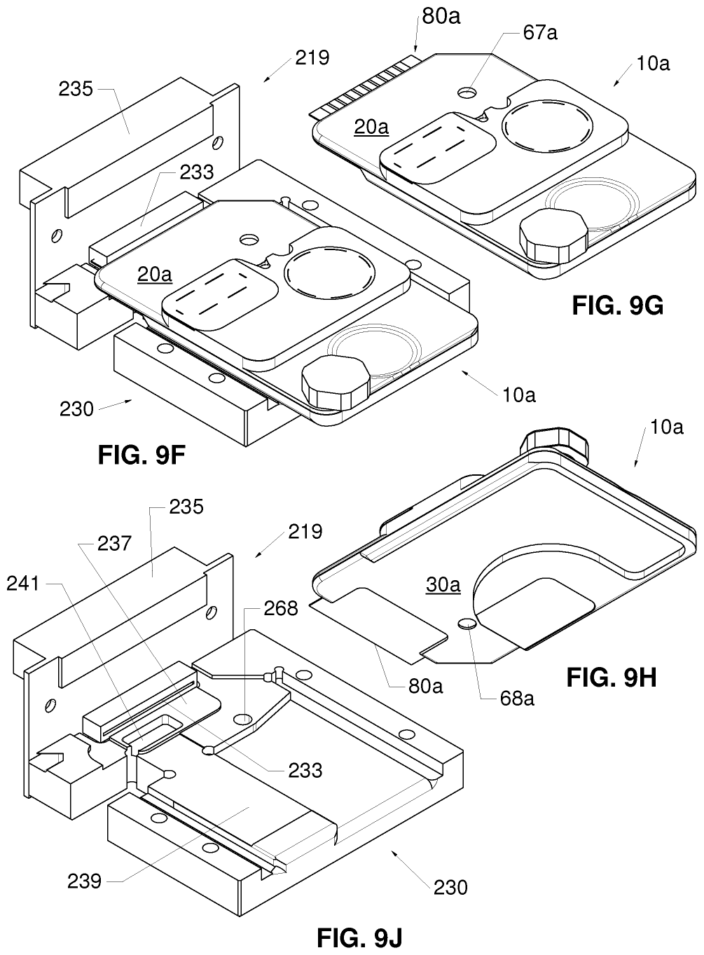

When a cartridge is inserted properly in the receptor of the analyzer, the cartridge biosensor electrical contact mates with the analyzer electrical contact (see FIGS. 9A-9J), bringing the optical chamber of the cartridge in position to receive EMR from the EMR source. Those skilled in the art will appreciate that the EMR could also be channeled to the optical chamber by optical fibers. The EMR transmitted through the blood sample in the cartridge, or reflected from the blood sample, impinges upon one or more photodetectors within the analyzer. Calibration algorithms for spectroscopic measurements are preferably installed within the processor of the analyzer, for transforming the spectroscopic signals into analyte measurements. Calibration algorithms for biosensor measurements are preferably installed within the processor of the analyzer, for transforming the biosensor signals into analyte measurements, but some biosensors require calibration prior to sample measurement. The measurements are usually in concentration units, but those skilled in the art will appreciate that other parameters can be measured, for example, without limitations, the ratio of the concentrations of two different analytes.

In some embodiments, the joint-diagnostic spectroscopic and biosensor analyzer further comprises a display screen for viewing the results and aiding the operator in use of the analyzer, as well as buttons for manipulating the display function. Those skilled in the art will appreciate that the analyzer could be connected to a host computer. Therefore, some embodiments of the system also comprise at least one communication port for interfacing with other instruments. Other non-limiting examples of other instruments are a printer, and diagnostic testing instruments like a pulse oximeter or some other non-invasive testing instrument. The optional communication port is also used to upgrade information in the analyzers processor, as well as to upload information from the analyzers processor. Another optional port in the housing of some embodiments of the joint-diagnostic spectroscopic and biosensor analyzer is provided for charging the power supply within the analyzer. Those skilled in the art will appreciate that a single port can be used for both data transfer and a power supply, for example, without any limitation, a USB (Universal Serial Bus) port. In some embodiments of a system, data transfer to and from the analyzer is accomplished by wireless means that are known by one of skill in the art, and therefore, for the sake of brevity, wireless communication means will not be discussed here.

Some embodiments of the joint-diagnostic spectroscopic and biosensor analyzer comprise one photodetector (photodiode), or more than one photodetector assembled as an array of detectors in a spectrometer, wherein the spectrometer comprises a grating for dispersing EMR emerging from the fluid sample, into wavelength components. The analyzer optionally comprises one or more focusing lenses between the disposable cartridge and the spectrometer. A person of ordinary skill in the art will appreciate that other forms of optical detection, for example, CCD (charged-coupled device), can be used, and are therefore considered to be within the scope of the invention.

In some embodiments, the interior walls of the cartridges are treated with a hydrophilic coating to promote even spreading of the blood within the optical chamber, and to promote movement of blood along the flow path by capillary action. An alternative to a hydrophilic coating is plasma or corona treatment of a surface for making the surface more hydrophilic.

The optical chamber is located along a flow path, and the optical chamber has at least one optical window for spectroscopic analysis of the blood. The at least one optical window is in alignment with at least a portion of the optical chamber. A flow path may also contain one or more reagents, anywhere along the flow path, for example, without limitation, an anticoagulant, a hemolyzing reagent, or a reagent that reacts with an analyte to enhance the absorbance of EMR. The optical chamber is specifically designed to reduce the average attenuation of EMR due to scattering of EMR by the intact red blood cells in a blood sample, without having to hemolyze the red blood cells using sound waves or hemolyzing chemicals. Preferably, the depth of the optical chamber, i.e., the internal distance between the optical windows, is in an approximate range of about 50 microns to about 200 microns. In a preferred embodiment, the depth of the optical chamber is substantially uniform across the optical windows. In some embodiments, the depth of the optical chamber is not uniform across the optical windows, and is within the scope of the present invention. A person of ordinary skill in the art will appreciate that although the optical windows are illustrated as circular elements, they can have other shapes, for example, without being limited, oval and square shapes. In some embodiments, the area of an optical window that is in alignment with an optical chamber is in an approximate range of about 1 sq. millimeter to about 100 sq. millimeters. For the sake of minimizing sample volume, a more preferred optical window area that is in alignment with the optical chamber is in an approximate range of about 1 sq. millimeter to about 10 sq. millimeters.

The biosensor conduit is located along a flow path, and the biosensor conduit may have one or more than one biosensors for analyzing the blood. Those skilled in the art will appreciate that biosensors may include various transducer arrangements that convert at least one property of the fluid sample into an electrical signal, wherein the transducer comprises at least one active surface for contacting the fluid sample. In some embodiments, the active surface is one of a chemical sensitive surface, or an ionic sensitive surface, and wherein the biosensor comprises at least one of a transistor, an ion-selective membrane, a membrane-bound enzyme, a membrane-bound antigen, a membrane-bound antibody, or a membrane-bound strand of nucleic acid. The disposable cartridge also comprises at least one biosensor electrical contact, and the cartridge receptor of the analyzer also comprises at least one analyzer electrical contact. Although the examples illustrated show the cartridge electrical output contact as flat pins in an array, those skilled in the art will appreciate that the electrical contacts can mate in other ways, for example, the electrical contacts described in U.S. Pat. No. 8,206,650.

Some embodiment of a joint-diagnostic spectroscopic and biosensor analyzer optionally comprises a barcode reader for reading a barcode on the disposable cartridge, the barcode containing at least information regarding calibration of a biosensor. The barcode also optionally contains information about the joint-diagnostic spectroscopic and biosensor analyzer. Some embodiments of disposable cartridges comprise radio frequency identification (RFID) tags. In some embodiments, the disposable cartridge further comprises a calibration fluid pouch containing a calibration fluid that is arranged in fluid connection with a biosensor conduit. For cartridges with calibration fluid pouches, the joint-diagnostic spectroscopic and biosensor system further comprises means for rupturing the calibration fluid pouches, for example, which should not be considered limiting in any way, a rotating cam, a reciprocating plunger, or a stepper motor linear actuator, and a spike in the cartridge housing. In some embodiments, the pouch itself contains an object with multiple spikes, which ruptures the calibration fluid pouch when pressure is applied to the calibration fluid pouch. In some embodiments, a portion of the seal of the calibration pouch is substantially weaker by design, than the rest of the seal, for easy rupture after pressure is applied. These weaker seal portions are sometimes referred to as frangible seals.

Some embodiments of cartridges also include at least one visible fill line or indicator serving as a marker providing a user with a visual indicator relating to the sufficiency of the blood sample in the optical chamber. Preferably the cartridge housing is made of transparent plastic for easy viewing of the blood inside the cartridge.

The means for calibrating the at least one biosensor includes a calibration fluid pouch 90 within the cartridge containing calibration fluid, means for rupturing the calibration pouch, and a calibration fluid conduit for transporting the calibration fluid from the pouch 90 to the biosensor conduit 54. U.S. Pat. No. 5,096,669 describes analyzer means for depressing and rupturing a calibration pouch. Although the cartridge embodiments shown comprise means for calibrating the biosensors, some cartridge embodiments have factory-calibrated biosensors, and therefore do not require means for calibrating the biosensors. These cartridge embodiments are also within the scope of the invention.

In some embodiments of a disposable cartridge, the blood storage conduit begins at a the blood storage conduit entrance and terminates at the optical chamber, and the volume of the blood storage conduit is in an approximate range of about 50 microliters to about 100 microliters. A small sample size is preferred for babies, but for pO₂ measurement, air bubbles can create greater errors in smaller samples. Therefore the size of the samples must be balanced between allowable errors and the amount of blood the patient can provide without causing the patient harm.

An air bladder is used for the purpose of urging blood along a path, and means for activating the air bladder is provided.

In some embodiments, the blood storage conduit has a length dimension measured from the proximal end (i.e., near the inlet) to the distal end (i.e., near the optical chamber) and has a cross-sectional area orthogonal to the length dimension, the size of the cross-sectional area being sufficiently small to receive the blood by capillary action, and the size being substantially uniform throughout a substantial portion of the length dimension. Cross-sectional areas are shown as semi-circular and rectangular, but a person with ordinary skill in the art will appreciate that other shapes can be used, and are therefore considered to be within the scope of the present invention.

The optical chamber of an embodiment of the cartridge has a depth dimension orthogonal to a plane of insertion of the cartridge into the receptor of the analyzer, wherein the depth dimension is in an approximate range of about 50 microns to about 200 microns. In the embodiments described in details later, the optical chamber is defined by a cut-out in the gasket 100. In some embodiments (not shown), the depth dimension of the optical chamber is greater than the thickness of the gasket.

Another aspect of an embodiment of a disposable cartridge (the first embodiment 10) for operation with a joint spectroscopic and biosensor blood analyzer for measurement of at least two hemoglobin species in blood by spectroscopy, and measurement of at least blood pH by biosensor, is a housing comprising: A) a first housing member 20; B) a second housing member 30; and C) a double-sided sticky gasket 100, are illustrated. U.S. Pat. No. 9,470,673 to the present inventor, the contents of which are hereby incorporated in entirety by reference, describes several other embodiments of the cartridge. Although the embodiments of a disposable cartridge illustrated in U.S. Pat. No. 9,470,673 comprise a single double-sided sticky gasket, some cartridge embodiments comprise more than two housing members, and therefore require more than one double-sided sticky gasket for bonding the additional housing members, for example, the second embodiment 10a.

In some embodiments of a cartridge, the double-sided sticky gasket has a thickness in the approximate range of about 50 microns to about 200 microns. Although the gaskets are described as sticky gaskets, non-sticky gaskets are considered within the scope of the invention. In embodiments using non-sticky gaskets, some form of adhesive is applied directly to the housing members at the areas where the gasket makes contact with the housing members, or some other means are used for sandwiching the gasket between the housing members.

The gaskets shown are flat and therefore each side of the gasket defines a plane, wherein both planes are parallel to each other. In some embodiments, the gasket is substantially flat, wherein each side substantially defines a plane, and wherein the two planes are not parallel. Therefore, it should be understood that reference to a plane orthogonal to the gasket means a plane orthogonal to either of the two planes substantially defined by the respective sides of a substantially flat gasket. As an example, a substantially flat gasket is one where most of the gasket is flat, but some sections comprise dimples and or bumps.

The gaskets illustrated comprise several gasket cut-outs but some embodiments of the cartridges comprise one or more than one gasket cut-out. As an example, consider gaskets 100 (according to a first embodiment of a cartridge) and 100a (according to a second embodiment of a cartridge) illustrated in cartridge embodiments 10 and 10a respectively, disclosed as examples. Cut-outs 101 and 102 in cartridge 10 are illustrated in cartridge 10a as a single cut-out labeled 101a, an embodiment vented through a groove in housing (not shown) does not have cut-out 107a; an embodiment comprising pre-calibrated biosensors (not shown) does not have cut-out 105a; and a cartridge embodiment in which there is no concern about contact between the calibration fluid and the gasket (not shown) does not have cut-out 106a. Moreover, in some embodiments cut-outs 101a is joined with cut-out 103a, and in some embodiments cut-out 103a is further joined to cut-out 104a (not shown). In order to minimize contact between the sample and the adhesive in the gasket some embodiments of cartridges comprise a single gasket cut-out, wherein a first portion of the cut-out is positioned to align with at least a portion of the optical chamber, and a second portion is positioned to at least align with the active area of the pH biosensor. In some embodiments, the single gasket is transformed into two separate gasket cut-outs: a first gasket cut-out is positioned to align with at least a portion of the optical chamber, and a second gasket cut-out is positioned to at least align with the active area of the pH biosensor. It is well known that adhesives are available that are compatible with the blood sample and the calibration fluids, and more cut-outs may be desired depending on the assembly process. Therefore at least one gasket cut-out is within the scope of the present invention.

With respect to spectroscopic measurements, those skilled in the art will appreciate the various ways a spectroscopic measurement apparatus can be constructed, and various elements that make up such apparatus. Accordingly, for the sake of brevity, description of basic spectroscopy and a list and function of the elements that make up a spectroscopic apparatus will not be discussed here. Those skilled in the art will appreciate that when the source of EMR is a single source, the single source could be split by a multi-channel optical fiber for providing more than one light paths. An example of a system for detecting the EMR transmitted through or reflected from a sample is an array of photodiodes, but those skilled in the art will appreciate that these spectroscopic elements are just examples and should not be considered limiting for the present invention.

Still with respect to spectroscopic measurements, the examples shown describe an apparatus that operates in transmission mode. Those skilled in the art will appreciate that the spectroscopic apparatus of a joint-diagnostic spectroscopic and biosensor analyzer can also operate in reflectance mode by placing a reflecting member in the analyzer receptor designed for receiving the cartridge, on one side of the optical chamber, such that the EMR transmitted through the sample would be reflected off the reflecting member, whereby the reflected EMR would enter the sample for the second time. In some embodiments of diagnostic measurement instruments or analyzers operating in the reflectance mode, both the EMR source and the photodetector are on the same side of the optical chamber. Moreover, those skilled in the art will also appreciate that instead of installing a reflecting member around the receptor in the housing of the analyzer, one side of the wall-portions of the optical chamber of the cartridge could be coated with a reflecting material.

A blood storage conduit is defined by a first blood storage conduit groove in one of the housing members, and either the gasket or the other housing member with or without a second blood storage conduit groove. In the embodiments where the blood storage conduit allows the blood to make contact with a surface of the gasket, the gasket is preferably made of hydrophilic material for enhancing wetting of the gasket, and the gasket adhesive is compatible with the sample. The blood storage conduit in some embodiments is simply a cut-out in the gasket with no grooves in either of the housing members. For clarity, the blood storage conduit in some embodiments, comprise a groove in the first housing member aligned with the gasket cut-out, or a groove in the second housing member alignment with the gasket cut-out. In yet other embodiments, the gasket cut-out is aligned with a first groove in the first housing member and a second groove in the second housing member. The illustration of the various embodiments of the blood storage conduit can be applied to other conduits, for example, the biosensor conduit, the blood shunt and the calibration fluid conduit, and are considered to be within the scope of the invention.

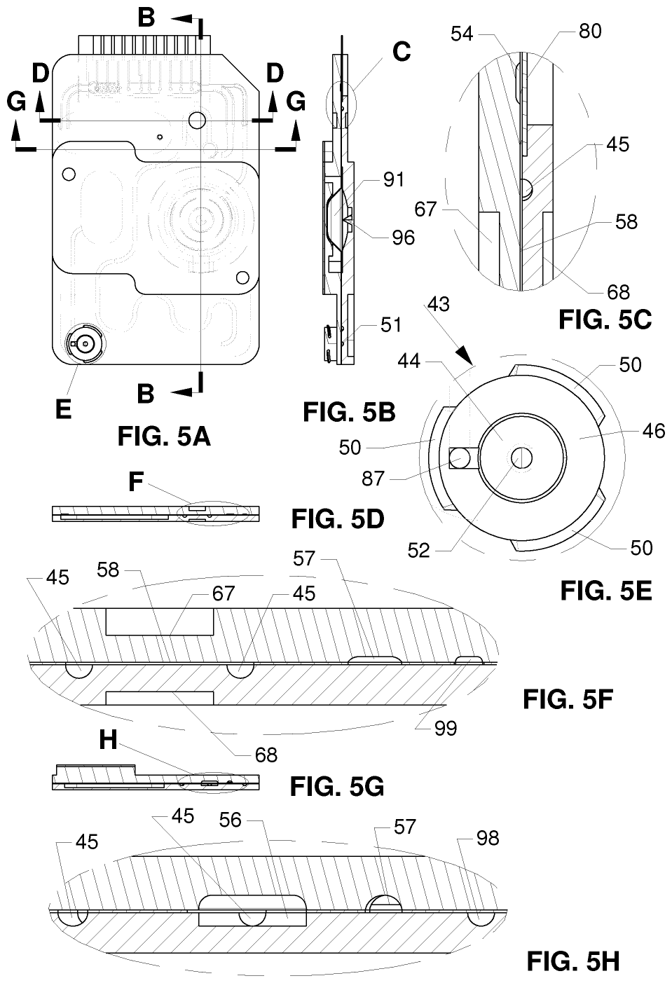

The housing of some embodiments of the disposable cartridge comprises a blood shunt (for example, 45 in FIG. 5H) for bypassing the optical chamber. The blood shunt provides fluid connectivity between the distal end of the blood storage conduit and the optical chamber overflow chamber, the blood shunt having a maximum bypass depth dimension orthogonal to the plane of insertion of the cartridge into the receptor of the analyzer, and wherein the maximum bypass depth dimension is substantially larger than the optical depth dimension, for enhancing blood flow from the distal end of the blood storage conduit to the biosensor conduit. The optical chamber overflow chamber refers to the general region in the blood flow path between the optical chamber 58 in cartridge 10 (and 58a in cartridge 10a) and the enlarged cavity 56 in cartridge 10 (and 56a in cartridge 10a).

The details of the drawings are discussed next, to further describe specific embodiments of the invention not completely described in U.S. Pat. No. 9,470,673, filed by the inventor. Two different cartridge embodiments are described in details, as examples only, and a person of ordinary skill in the art will appreciate that other embodiments that are not explicitly illustrated are implied. For easy reference, Table 1 provides a list of the reference numerals used, and a brief description of the structural features referred to. Attempts are made to use the same reference numerals for similar elements and, in some cases, the letter “a” is appended to the end of the number to refer to the second embodiment of the cartridge.

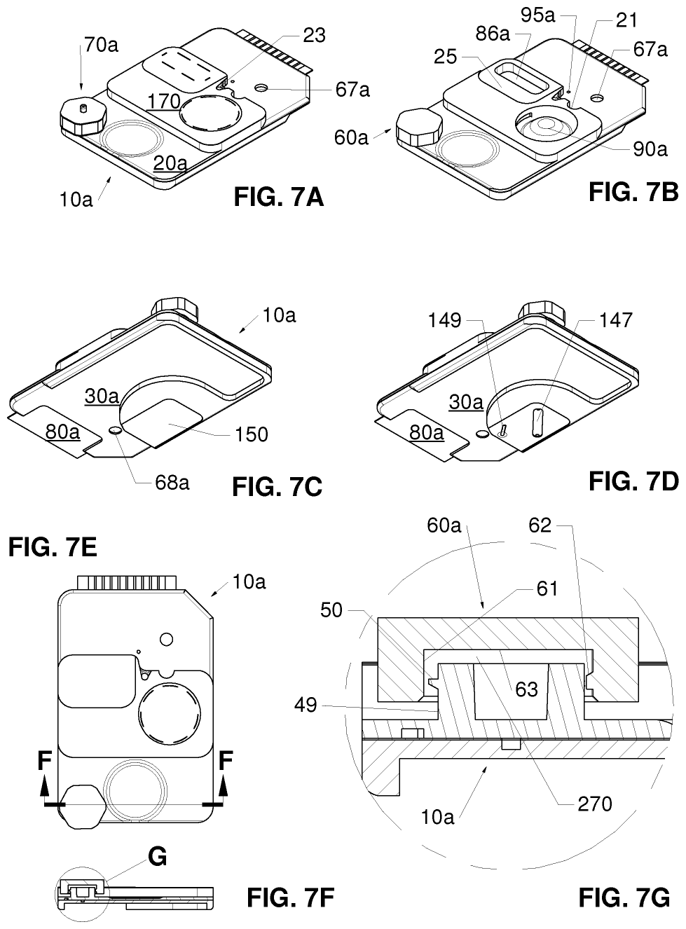

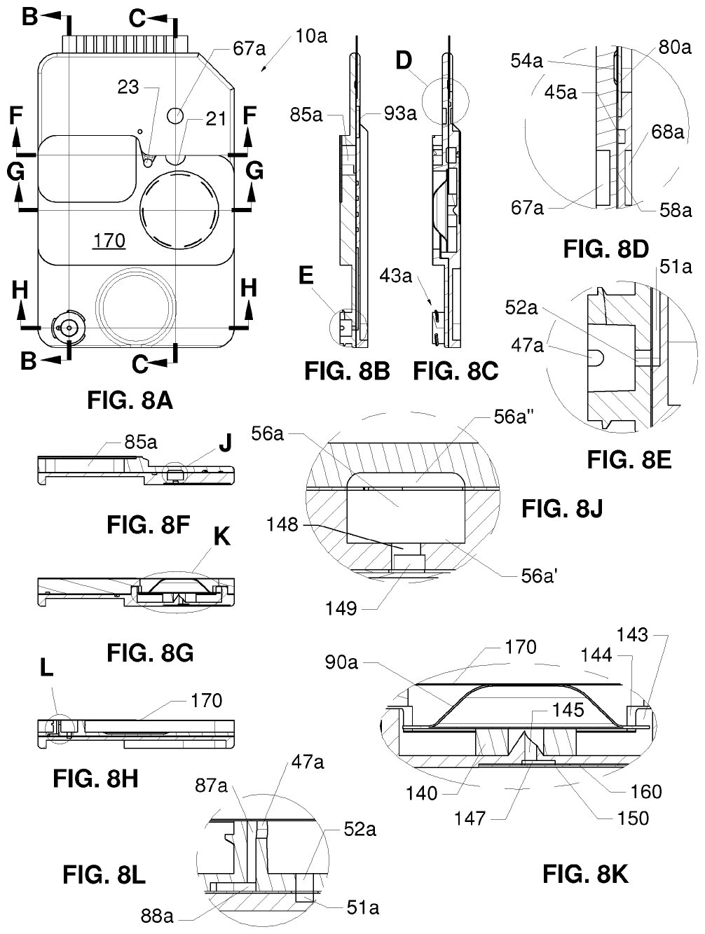

TABLE 1

| Reference | |
|---|---|
| Numerals | Description of Structural Features |
| 10 | Cartridge housing of a first embodiment of a cartridge |
| 10a | Cartridge housing of a second embodiment of a cartridge |
| 20 | First housing member of cartridge 10 |
| 20a | First housing member of cartridge 10a |
| 21 | Cut-out in 20a for viewing capillary break |
| 23 | Notch for receiving a spring-loaded cartridge locating pin 201 |
| | in cartridge receptor 200 |
| 25 | Air bladder recess for receiving flexible member 120 |
| 30 | Second housing member of cartridge 10 |
| 30a | Second housing member of cartridge 10a |
| 40 | Flexible member of cartridge 10 |
| 41 | Paddle in first housing member 20, for facilitating rupture of |
| | calibration fluid pouch |
| 42 | Paddle hinge in first housing member 20 |
| 43 | Cartridge inlet of cartridge 10 |
| 43a | Cartridge inlet of cartridge 10a |
| 44 | Cartridge inlet inner face surrounding blood storage conduit |
| | entrance 52 of cartridge 10 |
| 45 | Blood shunt for bypassing optical chamber 58 (formed by the |
| | distal end of the blood storage conduit groove 53 and the first |
| | housing member 20) |
| 45a | Blood shunt for bypassing optical chamber 58a (formed by |
| | the distal end of the blood storage conduit groove 53a and |
| | the first housing member 20a) |
| 46 | An annular surface at the top of the cartridge inlet 43 of |
| | cartridge 10 |
| 47 | Recess in the annular surface 46 of the cartridge inlet 43 |
| | cartridge 10 |
| 47a | Recess in the annular surface 46 of the cartridge inlet 43a |
| | cartridge 10a |
| 48 | Internal wall of the cartridge inlet 43 of cartridge 10 |
| 49 | External wall of the cartridge inlet 43 of cartridge 10 and inlet |
| | 43a of cartridge 10a |
| 50 | Cartridge inlet thread shown in this embodiment as |
| | abbreviated thread on external wall 49 of inlet 43 and 43a |
| 51 | Blood storage conduit of cartridge 10 |
| 51a | Blood storage conduit of cartridge 10a |
| 52 | Blood storage conduit entrance of blood storage conduit 51 |
| 52a | Blood storage conduit entrance of blood storage conduit 51a |
| 53 | Blood storage conduit groove of cartridge 10 |
| 53a | Blood storage conduit groove of cartridge 10a |
| 54 | Biosensor conduit of cartridge 10 |
| 54a | Biosensor conduit of cartridge 10a |
| 55 | Biosensor conduit groove of biosensor conduit 54 |
| 55a | Biosensor conduit groove of biosensor conduit 54a |
| 56′ | Portion of an enlarged cavity in first housing member 20 of |
| | cartridge 10 |
| 56a′ | Portion of an enlarged cavity in first housing member 20a of |
| | cartridge 10a |
| 56″ | Portion of an enlarged cavity in second housing member 30 |
| | of cartridge 10 |
| 56a″ | Portion of an enlarged cavity in second housing member 30a |
| | of cartridge 10a |
| 56 | Enlarged cavity of cartridge 10, comprising portions 56′, 56″, |
| | and a gasket cut-out 101 aligned with portions 56′ and 56″ |
| 56a | Enlarged cavity of cartridge 10a, comprising portions 56a′, |
| | 56a″, and a gasket cut-out 101a aligned with portions 56a′ |
| | and 56a″ |
| 57 | Connecting groove positioned to provide fluid connection |
| | between enlarged cavity 56 and biosensor conduit groove 55 |
| | of cartridge 10 |
| 57a | Connecting groove positioned to provide fluid connection |
| | between enlarged cavity 56a and biosensor conduit groove |
| | 55a of cartridge 10a |
| 58 | Optical chamber in cartridge 10a for receiving blood from |
| | blood storage conduit 51a, and positioned to align with at |
| | least a portion of an optical window |
| 58a | Optical chamber in cartridge 10a for receiving blood from |
| | blood storage conduit 51a, and positioned to align with at |
| | least a portion of an optical window |
| 60 | Cap for sealing cartridge inlet 43 of cartridge 10 |
| 60a | Cap for sealing cartridge inlet 43a of cartridge 10a |
| 61 | Internal wall surface of cap 60 and cap 60a |
| 62 | Cap thread, shown in this embodiment as abbreviated thread |
| | on internal wall 61 of cap 60 and cap 60a |
| 63 | Underside of cap 60 and cap 60a |
| 64 | Blind hole at roof of biosensor conduit groove 55 for trapping |
| | air |
| 65 | First through hole for facilitating assembly of cartridge |
| 66 | Second through hole for facilitating assembly of cartridge |
| 67 | First optical window of cartridge 10 |
| 67a | First optical window of cartridge 10a |
| 68 | Second optical window of cartridge 10 |
| 68a | Second optical window of cartridge 10a |
| 69 | Capillary adaptor handgrip for handling capillary adaptor 70 |
| 70 | Capillary adaptor for use with cartridge 10 |
| 70a | Capillary adaptor for use with cartridge 10a |
| 71 | Capillary adaptor inlet member comprising a capillary adaptor |
| | tube, of capillary adaptor 70 |
| 72 | Capillary adaptor inlet port of capillary adaptor 70 |
| 73 | Capillary adaptor outlet member of capillary adaptor 70 |
| 74 | Capillary adaptor outlet port of capillary adaptor 70 |
| 75 | Capillary adaptor face surrounding capillary adaptor outlet |
| | port 74 of capillary adaptor 70 |
| 76 | Capillary adaptor lumen of capillary adaptor 70 |
| 77 | Underside of capillary adaptor 70 |
| 78 | Internal wall of capillary adaptor 70 |
| 79 | Capillary adaptor thread, shown in this embodiment as |
| | abbreviated thread on internal wall 78 of capillary adaptor 70 |
| 80 | A biosensor array of cartridge 10, comprising at least a pH |
| | biosensor |
| 80a | A biosensor array of cartridge 10a, comprising at least a pH |
| | biosensor |
| 81 | Active area of biosensor array 80 |
| 82 | Biosensor electrical contact |
| 83 | Biosensor receptacle for arranging one or more biosensors in |
| | cartridge housing 10 |
| 84 | Bowl in nest 92 for receiving the flat side of the pouch 90 as it |
| | bulges under pressure |
| 85 | Air bladder of cartridge 10 |
| 85a | Air bladder of cartridge 10a |
| 86 | Air bladder cavity of cartridge 10 |
| 86a | Air bladder cavity of cartridge 10a |
| 87 | Air bladder exit port of cartridge 10 |
| 87a | Air bladder exit port of cartridge 10a |
| 88 | An air bladder conduit of cartridge 10 to provide fluid |
| | connection between air bladder and air bladder exit port 87 |
| 88a | An air bladder conduit of cartridge 10a to provide fluid |
| | connection between air bladder and air bladder exit port 87a |
| 89 | Air bladder conduit groove in first housing member 20 of |
| | cartridge 10 to provide fluid connection between air bladder |
| | and an air bladder exit port |
| 89a | Air bladder conduit groove in first housing member 20a of |
| | cartridge 10a to provide fluid connection between air bladder |
| | and an air bladder exit port |
| 90 | Calibration fluid pouch for storing and releasing calibration |
| | fluid, for cartridge 10 |
| 90a | Calibration fluid pouch for storing and releasing calibration |
| | fluid, for cartridge 10a |
| 91 | Calibration fluid pouch cavity of pouch 90 |
| 92 | Nest for receiving flat side of calibration fluid pouch 90 |
| 92a | Nest for receiving flat side of calibration fluid pouch 90a |
| 93 | Waste receptacle of cartridge 10 for receiving liquid waste |
| 93a | Waste receptacle of cartridge 10a for receiving liquid waste |
| 94 | Waste receptacle cavity of cartridge 10 for forming waste |
| | receptacle 93 |
| 94a | Waste receptacle cavity of cartridge 10a for forming waste |
| | receptacle |
| 95 | Waste receptacle vent for relieving pressure in waste |
| | receptacle 93 |
| 95a | Waste receptacle vent for relieving pressure in waste |
| | receptacle 93a |
| 96 | Calibration fluid pouch spike of cartridge 10 |
| 96a | Calibration fluid pouch spike of cartridge 10a |
| 97 | Calibration fluid pouch spike recess in bowl 84 for housing |
| | the spike 96 |
| 98 | Proximal end of calibration fluid groove of cartridge 10 for |
| | receiving calibration fluid from calibration fluid pouch |
| 99 | Distal end of calibration fluid groove for transferring |
| | calibration fluid from proximal end of calibration fluid groove |
| | 98 to biosensor conduit 54 |
| 100 | Double-sided sticky gasket of cartridge 10 for engaging |
| | members 20 and 30 |
| 100a | Double-sided sticky gasket of cartridge 10a for engaging |
| | members 20a and 30a |
| 101 | Gasket cut-out 101 positioned along blood storage conduit |
| | 51, optical chamber 58, and optical chamber overflow |
| | chamber up to the enlarged cavity 56 (see FIGS. 5C, 5F and |
| | 5H). |
| 101a | Gasket cut-out 101a positioned along blood storage conduit |
| | 51a, optical chamber 58a, and optical chamber overflow |
| | chamber up to the enlarged cavity 56a (see FIGS. 8D, 8E and |
| | 8J). |
| 102 | Gasket cut-out 102 positioned along portion of connecting |
| | groove 57 |
| 103 | Gasket cut-out 103 positioned to align with a portion of the |
| | biosensor conduit groove and the active area 81 of biosensor |
| | array 80 |
| 103a | Gasket cut-out 103a positioned to align with a portion of the |
| | biosensor conduit groove and the active area of biosensor |
| | array 80a |
| 104 | Gasket cut-out 104 positioned to provide fluid connection |
| | between distal end of biosensor conduit 54 and waste |
| | receptacle cavity 94 |
| 104a | Gasket cut-out 104a positioned to provide fluid connection |
| | between distal end of biosensor conduit 54a and waste |
| | receptacle cavity 94a |
| 105 | Gasket cut-out 105 positioned to align with calibration fluid |
| | pouch 90 |
| 105a | Gasket cut-out 105a positioned to align with calibration fluid |
| | pouch 90a |
| 106 | Gasket cut-out 106 positioned along portion of distal end of |
| | calibration fluid groove 99 |
| 106a | Gasket cut-out 106a positioned along portion of distal end of |
| | calibration fluid groove 99a |
| 107 | Gasket cut-out 107 positioned to align with waste receptacle |
| | vent 95 |
| 107a | Gasket cut-out 107a positioned to align with waste receptacle |
| | vent 95a |
| 120 | Flexible member of cartridge 10a for construction of air |
| | bladder |
| 130 | Double-sided sticky gasket for engaging flexible member 120 |
| | to member 40a |
| 140 | Annular compressible member surrounding spike 96a for |
| | supporting calibration fluid pouch 90a |
| 141 | Calibration fluid pouch window |
| 143 | Boss in second housing member 30a surrounding calibration |
| | fluid pouch 90a in cartridge 10a |
| 144 | Recess in first housing member 20a for receiving boss 143 |
| 145 | Hole in spike 96a fordraining calibration fluid to bottom side |
| | of second housing member 30a |
| 147 | Recess in bottom of member 30a for channelling calibration |
| | fluid |
| 148 | Hole in portion 56a′ of an enlarged cavity in second housing |
| | member 30a of cartridge 10a |
| 149 | Recess in bottom of second housing member 30a for |
| | channelling blood |
| 150 | Bottom cover of second housing member 30a of cartridge |
| | 10a |
| 160 | Double-sided sticky gasket for engaging bottom cover 150 to |
| | member 30a |
| 170 | Perforated label of cartridge 10a |
| 200 | Cartridge receptor |
| 201 | Spring-loaded cartridge locating pin |
| 203 | Air bladder depressor |
| 205 | Calibration fluid pouch depressor |
| 213 | Stepper motor for activating air bladder |
| 215 | Stepper motor for rupturing calibration fluid pouch |
| 216 | Power lines for stepper motors |
| 217 | Bracket mounted on cartridge receptor 200 for supporting |
| | stepper motors 213 and 215 |
| 218 | Back portion of receptor 200 |
| 219 | A physical interface attached to portion 218 of receptor 200 |
| | on one side and for connecting with an analyzer processor |
| 220 | Top portion of cartridge receptor 200 |
| 230 | Bottom portion of cartridge receptor 200 |
| 233 | Ports for facilitating electrical connection between biosensors |
| | 80a and physical interface 219 |
| 235 | Ports for facilitating electrical connection between physical |
| | interface 219 and processor |
| 237 | Bed for installing first heating element |
| 239 | Bed for installing second heating element |
| 241 | Cavity for housing a thermistor for regulating first and second |
| | heating elements |
| 245 | Shunt groove for defining blood shunt 45 for bypassing |
| | optical chamber 58 |
| 245a | Shunt groove for defining blood shunt 45a for bypassing |
| | optical chamber 58a |
| 248 | Hole in at the end of shunt groove 245a, traversing the |
| | thickness of the second housing member 30a of cartridge |
| | 10a, and fluidly connected to recess 149 |
| 267 | Opening in cartridge receptor 200 aligned with first optical |
| | window 67a of cartridge 10a |
| 268 | Opening in cartridge receptor 200 aligned with second optical |
| | window 68a of cartridge 10a |
| 270 | Cap airflow path, i.e,, a gap between external wall 49 of the |
| | cartridge inlet 43a of cartridge 10a, and Internal wall surface |
| | 61 of cap 60a, and between underside 63 of cap 60a and |
| | annular surface 46 at the top of the cartridge inlet 43a of |
| | cartridge 10a, before underside 63 makes contact with |
| | annular surface 46 (see FIG. 7G). |
| 300 | Microprocessor of analyzer |
| 310 | Source of electromagnetic radiation (EMR) |
| 320 | Spectrometer |
| 330 | EMR source circuit board |
| 340 | Spectrometer circuit board |
| 350 | Biosensor circuit board |
| 360 | Limit switch for notifying microprocessor that cartridge is |
| | fully inserted |
| 370 | Power supply |
| 380 | Heater controller |
| 390 | Stepper motor circuit board |
| 400 | Analyzer display screen |
| 410 | Analyzer printer |

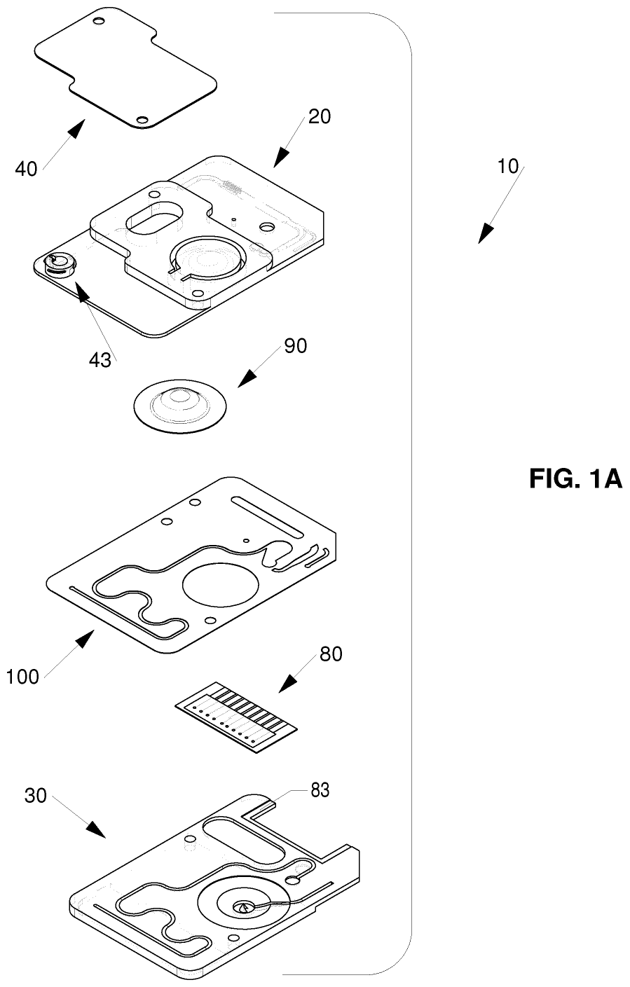

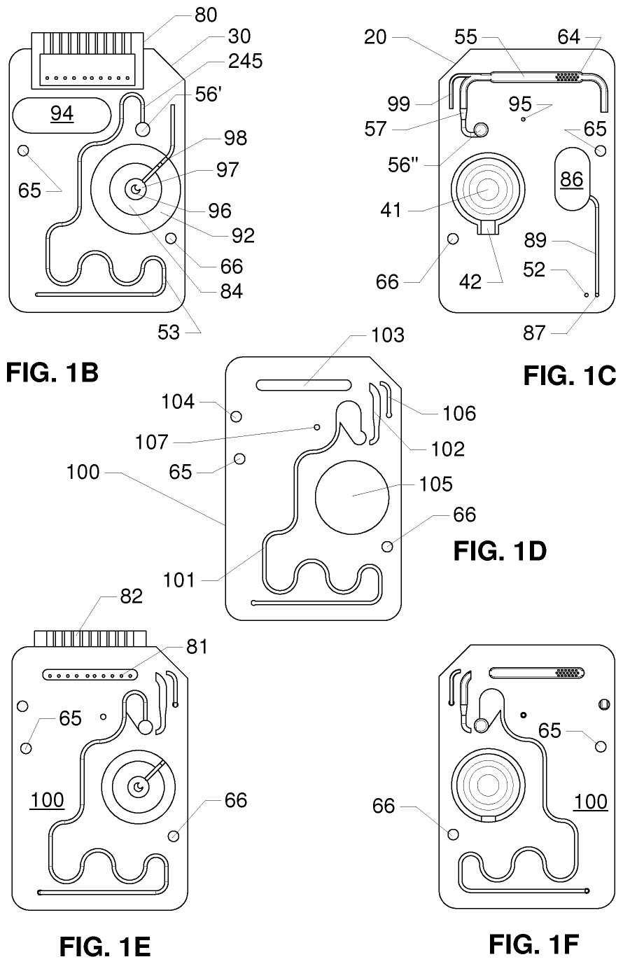

Shown in FIG. 1A is an exploded view of a first embodiment of a spectroscopic and biosensor cartridge 10. From top to bottom, components are listed as follows: a flexible member 40, a first housing member 20 showing a cartridge inlet 43, a calibration fluid pouch 90, a double-sided sticky gasket 100, a biosensor array 80, and a second housing member 30 showing a biosensor receptacle 83.

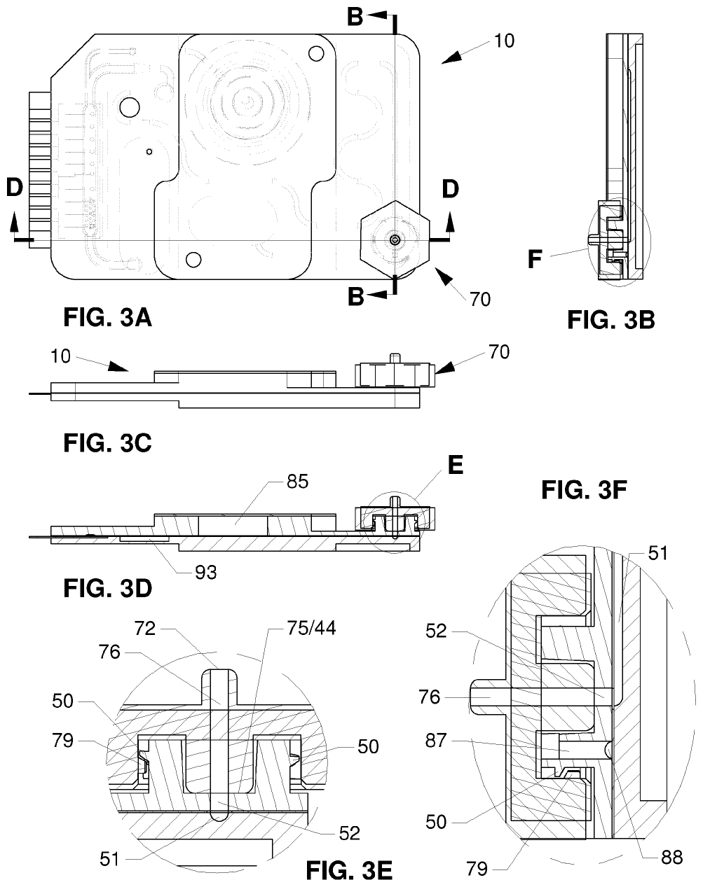

Shown collectively in FIGS. 1B-1F are more details of the components of the cartridge. FIG. 1B illustrates a top view of the second housing member 30 of the cartridge, with the biosensor array 80 installed in the receptacle 83 shown in FIG. 1A. Also shown in FIG. 1B are a nest 92 for receiving the flat side of the calibration fluid pouch 90 (hidden), a bowl 84 in nest 92 for receiving the flat side of the pouch 90 as it bulges under pressure, a calibration fluid pouch spike recess 97 in bowl 84 for housing a spike 96, the proximal end of a calibration fluid groove 98 for receiving calibration fluid from calibration fluid pouch 90 after it is ruptured by the spike 96, a first through hole 65 and a second through hole 66 for facilitating assembly of the cartridge, and a waste receptacle cavity 94 for forming a waste receptacle 93 illustrated in FIG. 3D.

FIG. 1D illustrates the gasket 100 having several cut-outs described in Table 1. As already explained, a single gasket cut-out is considered to be within the scope of the present invention. FIG. 1E and FIG. 1F illustrate how the gasket 100 is aligned with the second housing member 30 and the first housing member 20 respectively. FIG. 1E illustrates a top view of the second housing member 30 shown in FIG. 1B, overlaid by and in alignment with the gasket 100 shown in FIG. 1D, FIG. 1F illustrates a bottom view of the first housing member 20 shown in FIG. 1C, overlaid by and in alignment with the gasket 100 shown in FIG. 1D.

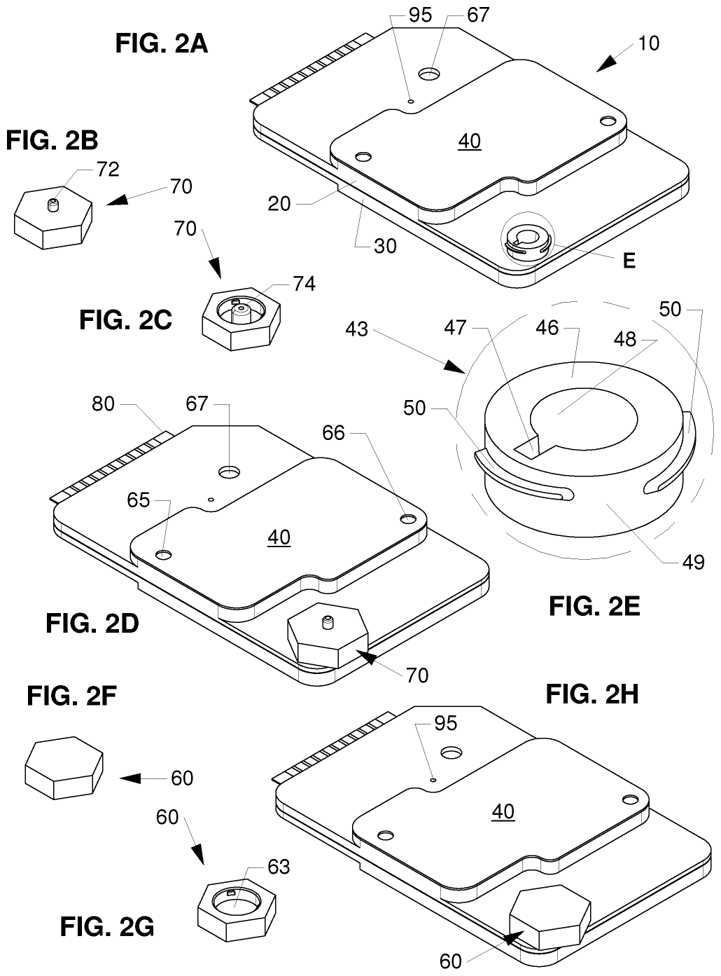

Illustrated in FIG. 2A is a perspective view of the joint-diagnostic spectroscopic and biosensor cartridge 10 shown in FIG. 1A, showing the first housing member 20, the second housing member 30, the flexible member 40, which covers the paddle 41 and paddle hinge 42, as well as the air bladder cavity 86 (see FIG. 1C). Flexible member 40 is stuck on to the first housing member 20, so as to create the air bladder 85 (see FIG. 3D), and seal off the nest 92 in order to direct calibration fluid from a ruptured pouch 90 into the proximal end of the calibration fluid groove 98. Also shown in FIG. 2A is waste receptacle vent 95 for relieving pressure in waste receptacle 93, a first optical window 67, and a cartridge inlet 43 (detail E shown in FIG. 2E). Illustrated in FIG. 2B is a first perspective view of an embodiment of a capillary adaptor 70 for use with cartridge 10 shown in FIG. 2A, showing the capillary adaptor inlet port 72. Illustrated in FIG. 2C is a second perspective view of the capillary adaptor 70 shown in FIG. 2B, showing the capillary adaptor outlet port 74. Illustrated in FIG. 2D is the capillary adaptor 70 shown in FIG. 2B, engaged with the inlet 43. Details of inlet 43 are illustrated in FIG. 2E, showing an annular surface 46, a recess 47 in the annular surface 46, an internal wall 48, external wall 49, and thread 50 on external wall 49.

Illustrated in FIG. 2F is a first perspective view of an embodiment of a cap 60 for use with cartridge 10 shown in FIG. 2A. Illustrated in FIG. 2G is a second perspective view of the cap 60 shown in FIG. 2F, showing the underside 63 of the cap. Illustrated in FIG. 2H is the cap 60 shown in FIG. 2F, engaged with the inlet 43 of the cartridge 10 shown in FIG. 2A. Some embodiments of a cap comprise a gasket attached to the underside 63, for creating an air-tight seal between the underside 63 of the cap and the annular surface 46 at the top of the cartridge inlet 43.

Illustrated in FIG. 3A is a top view of the cartridge and capillary adaptor engaged as shown in FIG. 2D. Illustrated in FIG. 3B is a first cross-sectional view through the cartridge and capillary adaptor shown in FIG. 3A along line B-B. Illustrated in FIG. 3C is a front view of the cartridge and capillary adaptor shown in FIG. 3A. Illustrated in FIG. 3D is a second cross-sectional view through the cartridge and capillary adaptor shown in FIG. 3A along line D-D, showing the air bladder 85 and the waste receptacle 93.

Illustrated in FIG. 3E is a detailed view of the detail E of the cartridge and capillary adaptor shown in FIG. 3D, showing capillary adaptor inlet port 72, capillary adaptor lumen 76, blood storage conduit entrance 52, blood storage conduit 51, interface 75/44 of the cartridge inlet inner face 44 and the capillary adaptor face 75, abbreviated thread 50 on external wall 49 of inlet 43, abbreviated thread 79 on internal wall 78 of capillary adaptor 70. In this embodiment, the cartridge inlet inner face 44 and the capillary adaptor face 75 are shown mating by design, and also by design, annular surface 46 of the cartridge inlet 43 does not mate with underside 77 of the capillary adaptor 70.

Also illustrated in FIG. 3E in conjunction with FIG. 2E is the internal wall 48 (see FIG. 2E) defining an airflow path for airflow between an exterior of the disposable cartridge and the blood storage conduit 51 when the capillary adaptor is being removed from the cartridge inlet 43. The airflow path in some embodiments is defined by one or more than one flute or groove in the internal wall 48 of the inlet 43, or in the external wall of the capillary adaptor outlet member 73. In such embodiments, there is frictional engagement between the internal wall 48 of the inlet 43 and the capillary adaptor outlet member 73. In the embodiment illustrated in FIG. 3E, capillary adaptor outlet member 73 could be inserted into cartridge inlet 43 with no frictional engagement. Since there is no seal between capillary adaptor outlet member 73 and the internal wall 48 of the cartridge inlet 43, the fitting of the present invention is not characterized as a standard Luer fitting. Moreover, a standard Luer fitting is not compatible with the present invention as explained next. There are two types of Luer fittings: Luer slip and Luer lock. A Luer slip fitting consists of a tapered cone and a mating tapered cavity. A Luer lock fitting consists of a Luer slip fitting with locking threads added. The Luer lock fitting creates a more secure connection to the Luer slip connection. If the present invention was a standard Luer lock fitting, two situations would occur: 1) there would have been a gap between the cartridge inlet inner face 44 and the capillary adaptor face 75—such a gap would impede capillary flow of blood into the blood storage conduit 51, and is not preferred; 2) removal of the male member (in this case the capillary adaptor outlet member 73) would have created a vacuum—withdrawing the blood in the blood storage conduit 51, away from the optical chamber, and attempt to refill the optical chamber has the potential to create air bubbles in the optical chamber. The inlet wall 48 also defines a similar airflow path for airflow between an exterior of the disposable cartridge and the blood storage conduit 51 when a syringe containing blood is used to fill the blood storage conduit 51, instead of a capillary adaptor, when the syringe is being removed from the cartridge inlet 43.

Illustrated in FIG. 3F is a detailed view of the detail F of the cartridge and capillary adaptor shown in FIG. 3B showing blood storage conduit entrance 52, blood storage conduit 51, capillary adaptor lumen 76, air bladder conduit 88, air bladder exit port 87, abbreviated thread 50 on external wall 49 of inlet 43, and abbreviated thread 79 on internal wall 78 of capillary adaptor 70.

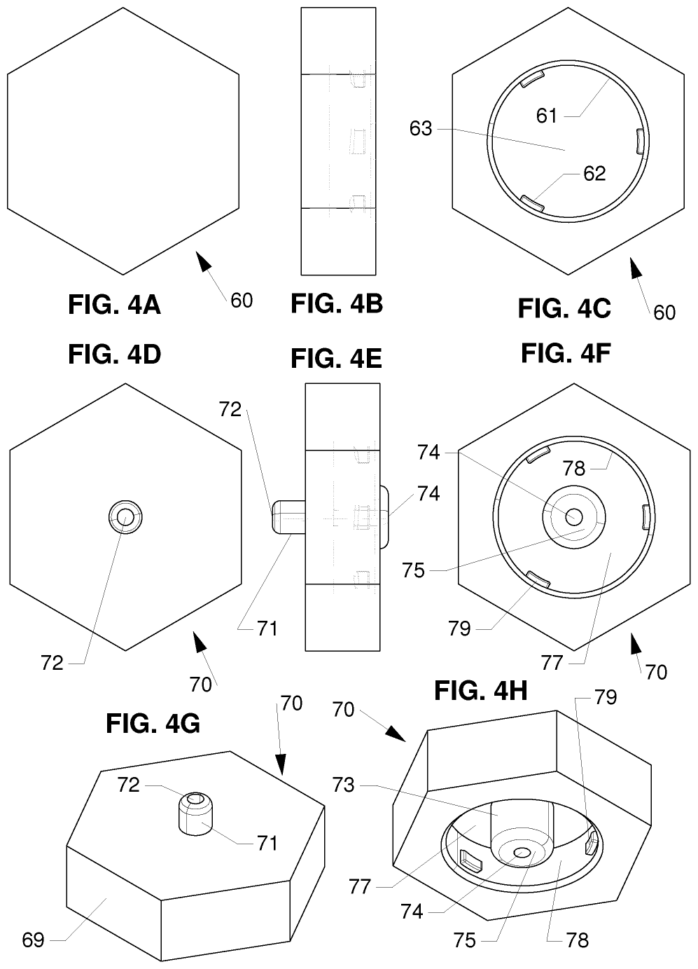

Illustrated in FIG. 4A is a top view of the cap 60 shown in FIG. 2F. Illustrated in FIG. 4B is a right side view of the cap shown in FIG. 4A. Illustrated in FIG. 4C is a bottom view of the cap shown in FIG. 4A, showing underside 63 of cap 60, internal wall surface 61 of cap 60, and abbreviated thread 62 on internal wall 61 of cap 60.

Illustrated in FIG. 4D is a top view of the capillary adaptor 70 shown in FIG. 2B. Illustrated in FIG. 4E is a right side view of the capillary adaptor shown in FIG. 4D, showing capillary adaptor inlet member 71, capillary adaptor inlet port 72, and capillary adaptor outlet port 74. Illustrated in FIG. 4F is a bottom view of the capillary adaptor shown in FIG. 4D showing capillary adaptor outlet port 74, capillary adaptor face 75 surrounding capillary adaptor outlet port 74, underside 77 of capillary adaptor, and internal wall 78 of capillary adaptor 70.

Illustrated in FIG. 4G is a first perspective view of the capillary adaptor 70 shown in FIG. 4D, showing the capillary adaptor handgrip 69, a capillary adaptor inlet member 71 comprising a capillary adaptor tube and a capillary adaptor inlet port 72. Illustrated in FIG. 4H is a second perspective view of the capillary adaptor shown in FIG. 4D, showing in addition, a capillary adaptor outlet member 73, a capillary adaptor outlet port 74, a capillary adaptor face 75 surrounding the capillary adaptor outlet port 74, underside 77 and internal wall 78 of capillary adaptor 70.

Illustrated in FIG. 5A is a top view of the cartridge shown in FIG. 1A. Illustrated in FIG. 5B is a first cross-sectional view through the cartridge shown in FIG. 5A along line B-B, showing calibration fluid pouch cavity 91 of pouch 90, and calibration fluid pouch spike 96. Illustrated in FIG. 5C is a detailed view of the detail C of the cartridge shown in FIG. 5B, showing biosensor conduit 54, blood shunt 45, biosensor array 80, optical chamber 58, first optical window 67, and second optical window 68.

Illustrated in FIG. 5D is a second cross-sectional view through the cartridge shown in FIG. 5A along line D-D. Illustrated in FIG. 5E is a detailed view of the detail E of the cartridge shown in FIG. 5A, showing an annular surface 46, an abbreviated thread 50, a blood storage conduit entrance 52, and an air bladder exit port 87.

Illustrated in FIG. 5F is a detailed view of the detail F of the cartridge shown in FIG. 5D, showing blood shunt 45, optical chamber 58, first optical window 67, second optical window 68, connecting groove 57 positioned to provide fluid connection between enlarged cavity 56 and biosensor conduit groove 55, and distal end of calibration fluid groove 99 for transferring calibration fluid from proximal end of calibration fluid groove 98 to biosensor conduit 54.

Illustrated in FIG. 5G is a third cross-sectional view through the cartridge shown in FIG. 5A along line G-G. Illustrated in FIG. 5H is a detailed view of the detail H of the cartridge shown in FIG. 5G, showing blood shunt 45, enlarged cavity 56, connecting groove 57 positioned to provide fluid connection between enlarged cavity 56 and biosensor conduit groove 55, and proximal end of calibration fluid groove 98 for receiving calibration fluid from calibration fluid pouch 90.

All the previous Figures are illustration of a first embodiment of a cartridge 10, and all subsequent Figures are illustration of various aspects of a second embodiment of a cartridge 10a, FIGS. 9A-9J also illustrate aspects of an embodiment of a cartridge receptor 200. Attempts are made to use the same reference numerals for similar elements and, in some cases, the letter “a” is appended to the end of the number to indicate that the embodiment of the cartridge is the second embodiment. The differences between the first and second embodiments are highlighted, to illustrate various advantageous features over the prior art. Table 1 provides a list of the reference numerals used, and a brief description of the structural features referred to.

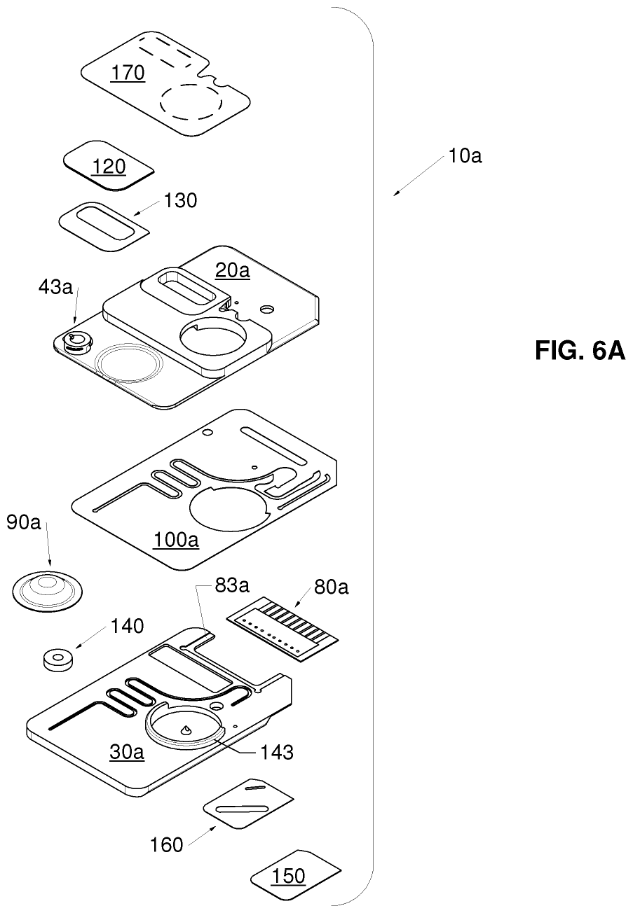

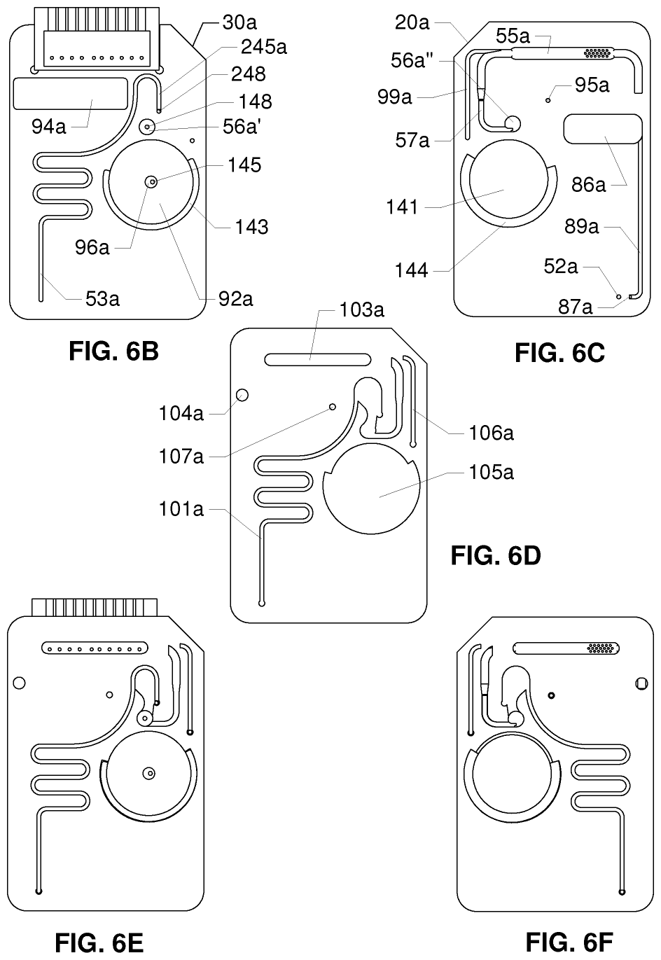

Illustrated in FIG. 6A is an exploded view of a second embodiment of a joint-diagnostic spectroscopic and biosensor cartridge 10a for use with a joint-diagnostic spectroscopic and biosensor analyzer. The major features in cartridge 10a that are not present in cartridge 10 are as follows: 1) the flexible member 120 only covers the air bladder cavity 86a (see FIG. 7B), and is attached to the first housing member 20a facilitated by a double-sided sticky gasket 130; 2) the calibration fluid pouch is only covered with a perforated label 170 (see FIG. 7A); 3) the calibration fluid pouch is only supported in the cartridge by an annular compressible member 140, which surrounds spike 96a; 4) the spike 96a comprises a hole 145 for draining calibration fluid to recess 147 in the bottom side of second housing member 30a for channeling the calibration fluid (see FIGS. 6B and 7D); 5) the blood shunt 45a (shown in FIG. 8D) fluidly connects with a recess 149 in second housing member 30a (shown in FIG. 7D) via a hole 248 (shown in FIG. 6B) in the second housing member 30a, and re-enters the enlarged cavity 56a from the bottom through a hole 148 in portion 56a′ of the enlarged cavity, in second housing member 30a (see FIG. 8J); 6) a preferably hard plate 150 covers the recesses 147 and 149 (see FIG. 7C) facilitated by a double-sided sticky gasket 160; 7) a boss 143 in second housing member 30a surrounds calibration fluid pouch 90a by greater than 180 degrees, and during cartridge assembly, positions the calibration fluid pouch 90a, which is supported by the annular compressible member 140; and 8) a recess 144 (see FIG. 6C) in the first housing member 20a, for receiving boss 143. A boss 143 surrounding the pouch 90a by greater than 180 degrees is as effective as 360 degrees, in preventing the pouch 90a from horizontal movement.

The advantage of the rupture mechanism described for cartridge embodiment 10a for releasing calibration fluid, over the rupture mechanism described for cartridge embodiment 10 and the embodiments described in U.S. Pat. No. 9,470,673, is the decreased force required to rupture the calibration fluid pouch, by direct observation.

The advantage of the fluid connection between the shunt 45a and the enlarged cavity 56a described for cartridge embodiment 10a for slowing down blood flow by capillary action, over the fluid connection between the shunt 45 and the enlarged cavity 56 described for cartridge embodiment 10 for slowing down blood flow by capillary action for cartridge embodiment 10 and the embodiments described in U.S. Pat. No. 9,470,673, is the efficiency of enlarged cavity 56a as a capillary break. FIGS. 6B and 8J collectively illustrate the progression from a small cross-sectional area of hole 148 to a substantially larger cross-sectional area of enlarged cavity 56a. In an analogous configuration described for cartridge embodiment 10 and in the embodiments described in U.S. Pat. No. 9,470,673, the progression from small to large is only in one dimension; this is best illustrated collectively in FIGS. 1B and 5H. In FIG. 1B, shunt groove 245 is connected to the side of enlarged cavity portion 56′; in FIG. 6B, the shunt groove 245a is effectively connected to the bottom of enlarged cavity portion 56a′, and the connection is illustrated in FIG. 6B as concentric circles. By direct observation, cartridge embodiment 10a provides a more effective capillary break than the capillary breaks in the embodiments described in U.S. Pat. No. 9,470,673. In cartridge 10a of the present invention, the enlarged cavity 56a is simultaneously in fluid connection with the optical chamber 58a and the blood shunt 45a via the hole 148 in portion 56a′ of an enlarged cavity.

Illustrated in FIG. 6B is a top view of the second housing member 30a of the cartridge, with the biosensor array 80a installed in the receptacle 83a, shown in FIG. 6A. Illustrated in FIG. 6C is a bottom view of the first housing member 20a of the cartridge shown in FIG. 6A. Illustrated in FIG. 6D is a top view of gasket 100a of the cartridge shown in FIG. 6A. Illustrated in FIG. 6E is the top view of the second housing member 30a shown in FIG. 6B, overlaid by and in alignment with the gasket 100a shown in FIG. 6D. Illustrated in FIG. 6F is the bottom view of the first housing member 20a shown in FIG. 6C, overlaid by and in alignment with the gasket 100a shown in FIG. 6D.

Illustrated in FIG. 7A is a perspective view of the joint-diagnostic spectroscopic and biosensor cartridge 10a shown in FIG. 6A, showing the perforated label 170 attached to the first housing member 20a, and a capillary adaptor 70a engaged with the inlet 43a. The perforations are for ease of breaking the label, when stepper motors 213 and 215 (see FIG. 9A) respectively, activate the air bladder and pushes the calibration fluid pouch 90a against the spike 96a. The punched-out portions of the label sticks to the flexible member of the air bladder and the dome of the calibration fluid pouch, and do not fall inside the receptor 200 of the analyzer.

Illustrated in FIG. 7E is a top view of cartridge 10a, with cap 60a partly engaged with the inlet 43a, and illustrated in FIG. 7F is a cross-sectional view through the cartridge and cap shown in FIG. 7E along line F-F. In order to illustrate a cap air flow path 270, i.e, a gap between external wall 49 of the cartridge inlet 43a of cartridge 10a, and Internal wall surface 61 of cap 60a, and between underside 63 of cap 60a and annular surface 46 at the top of the cartridge inlet 43a of cartridge 10a, before underside 63 makes contact with annular surface 46, FIG. 7G is provided. Illustrated in FIG. 7G is a detailed view of the detail G of the cartridge and cap shown in FIG. 7F. FIG. 7G viewed in conjunction with FIGS. 2E and 4C, illustrate the abbreviated threads 50 and 62. These abbreviated threads assist in providing the cap air flow path 270. In some embodiments, a cap absent threads is frictionally engaged with the external wall 49 of the cartridge; in such an embodiment, at least the inside wall of the cap or the external wall of the inlet comprise one or more flute or groove (not shown), to provide the cap air flow path 270.

Illustrated in FIG. 7B is a second perspective view of cartridge 10a shown in FIG. 7A, with air bladder cavity 86a and calibration fluid pouch 90a exposed by hiding perforated label 170, and a cap 60a engaged with the inlet 43a. The disposable cartridges illustrated in U.S. Pat. No. 9,470,673 comprise a hinged-paddle, which is absent in FIG. 7B. Removal of the hinged-paddle contributes to decreased force requirement for rupturing the pouch 90a.

Illustrated in FIG. 7C is a third perspective view of the joint-diagnostic spectroscopic and biosensor cartridge 10a shown in FIG. 7A, with the second housing member 30a exposed, showing a bottom cover 150. Illustrated in FIG. 7D is a fourth perspective view of cartridge 10a shown in FIG. 7A, with recesses 147 and 149 in the bottom of second housing member 30a exposed by hiding the bottom cover 150. Preferably, the cover 150 is made of hard material for strength, and has a small thickness in order to minimize the cartridge thickness.

Illustrated in FIG. 8A is a top view of the cartridge shown in FIG. 6A, showing a notch 23 for receiving a spring-loaded cartridge locating pin 201 in cartridge receptor 200, illustrated in FIG. 9D. Also shown is a cut-out 21 in 20a for viewing capillary break, in order to observe that blood has reached the entrance of the enlarged cavity 56a. Illustrated in FIG. 8B is a first cross-sectional view through the cartridge shown in FIG. 8A along line B-B. Illustrated in FIG. 8C is a second cross-sectional view through the cartridge shown in FIG. 8A along line C-C. Illustrated in FIG. 8D is a detailed view of the detail D of the cartridge shown in FIG. 8C, illustrating that the width of the blood shunt 45a is substantially larger than the width of the optical chamber 58a. Also shown is the biosensor conduit 54a.

Illustrated in FIG. 8E is a detailed view of the detail E of the cartridge shown in FIG. 8B. Illustrated in FIG. 8F is a third cross-sectional view through the cartridge shown in FIG. 8A along line F-F. Illustrated in FIG. 8G is a fourth cross-sectional view through the cartridge shown in FIG. 8A along line G-G. Illustrated in FIG. 8H is a fifth cross-sectional view through the cartridge shown in FIG. 8A along line H-H. Illustrated in FIG. 8J is a detailed view of the detail J of the cartridge shown in FIG. 8F. When FIG. 8J is viewed in conjunction with FIG. 6B, it is illustrated that the enlarged cavity 56a has a second cross-sectional area substantially larger than the cross-sectional area of the hole 148, whereby blood flow by capillary action slows down as the blood reaches the hole 148, and wherein the enlarged cavity 56a is simultaneously in fluid connection with the optical chamber 58a and the blood shunt 45a via the hole 148. It was observed that this arrangement provides a more effective capillary break that the capillary breaks described in U.S. Pat. No. 9,470,673.

Illustrated in FIG. 8K is a detailed view of the detail K of the cartridge shown in FIG. 8G, illustrating the annular compressible member 140 surrounding spike 96a for supporting calibration fluid pouch 90a, and a hole 145 in the spike 96a and in fluid connection with a recess 147 (see FIG. 7D). U.S. Pat. No. 9,470,673 does not disclose a hole in the spike, and it was observed that the present arrangement decreases the force required to rupture the pouch 90a. Although the compressible member 140 is illustrated as annular in shape, other shapes, for example, square and triangular, are considered to be within the scope of the present invention.

Illustrated in FIG. 8L is a detailed view of the detail L of the cartridge shown in FIG. 8A. Illustrated in conjunction with FIGS. 8A and 8E, is the association of the air bladder exit port 87a and the blood storage conduit 51a.

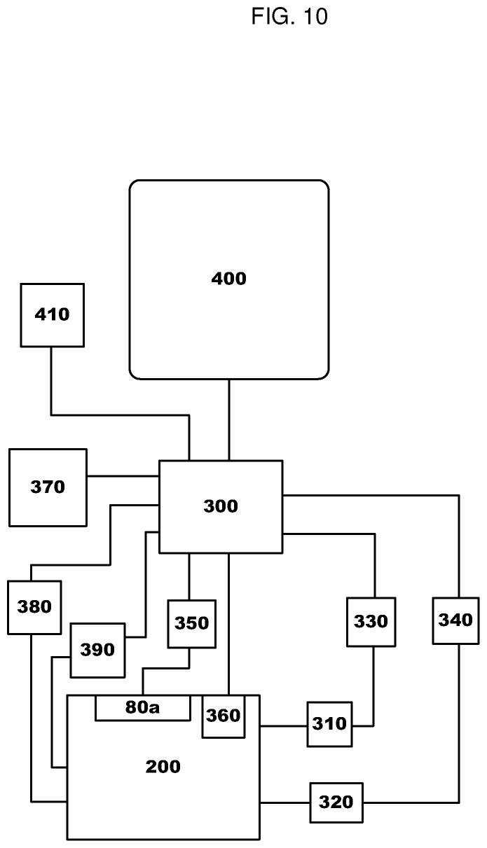

Illustrated collectively in FIGS. 9A-10 is an embodiment of a joint spectroscopic and biosensor system for measurement of at least two hemoglobin species in a patient's blood sample by spectroscopy, and measurement of at least pH of the blood sample by biosensor, for assessing the patient's oxygenation and acid-base status, comprising: a) an analyzer; b) a disposable cartridge 10a, and c) a cap for sealing the cartridge inlet 43a.

A block diagram of an embodiment of an analyzer for measurement of at least two hemoglobin species in a patient's blood sample by spectroscopy, and measurement of at least pH of the blood sample by biosensor, for assessing the patient's oxygenation and acid-base status, is provided in FIG. 10. The analyzer comprises a housing illustrated in U.S. Pat. No. 9,470,673 in FIGS. 14A-14C, which does not disclose details of the analyzer components. The housing comprises some of the following components: i) a power supply, which is optionally in the form of disposable or rechargeable batteries; ii) a source of electromagnetic radiation (EMR), for example, one or more LEDs, a tungsten lamp, one or more lasers, or any combination thereof; iii) a receptor in the analyzer housing for receiving a disposable cartridge; iv) a photodetector for measuring EMR transmitted through or reflected from a blood sample within the optical chamber of the cartridge and for providing an EMR-based signal derived from the EMR transmitted through or reflected from the blood sample; v) a processor or microprocessor for controlling the analyzer and in communication with the photodetector for receiving the EMR-based signal, and at least one calibration algorithm installed in the processor for transforming the EMR-based signal into a hemoglobin specie concentration; vi) a physical interface attached to the receptor for connecting with an analyzer processor and for connecting with the biosensor; vii) means for releasing the calibration fluid from the calibration fluid pouch and transporting released calibration fluid to the biosensor conduit for calibrating at least the pH biosensor prior to measuring the pH of the blood sample; viii) means for maintaining the active area of the biosensor at a pre-determined temperature; ix) means for preheating the blood sample; x) a display screen; and xi) a barcode reader. An optional printer is included in FIG. 10.

Illustrated in FIG. 9A is a perspective view of an embodiment of a joint-diagnostic spectroscopic and biosensor cartridge inserted in the receptor 200 of an analyzer. Illustrated in FIG. 9B is a top view of a joint-diagnostic spectroscopic and biosensor cartridge inserted in the receptor of an analyzer. Illustrated in FIG. 9C is a first cross-sectional view through the cartridge shown in FIG. 9B along line C-C. Illustrated in FIG. 9D is a second cross-sectional view through the cartridge shown in FIG. 9B along line D-D. Illustrated in FIG. 9E is a third cross-sectional view through the cartridge shown in FIG. 9B along line E-E.

Referring collectively to FIGS. 9A-9E, the analyzer receptor 200 is illustrated, comprising: a) an opening for receiving and aligning a capped disposable cartridge 10a containing the patient's blood in the cartridge, in an operational position (the opening refers to the front portion of receptor 200, occupied by the cartridge 10a), b) an opening 267 (or 268) for directing the electromagnetic radiation to the first optical window when the cartridge is in the operational position; c) and an opening 268 (or 267) for directing electromagnetic radiation emerging from the optical chamber to the at least one photodetector when the cartridge is in the operational position; d) a physical interface 219 for facilitating electrical connection with the biosensor electrical output element(s) and the processor; and e) a bracket 217 mounted on the receptor for at least supporting a stepper motor 215 for applying force to the calibration fluid pouch against a spike, and a stepper motor 213 for forcing air from the air bladder 85a through the air bladder exit port, for pushing the blood into the biosensor conduit. In some embodiments, the opening defines at least one surface for cooperating with a corresponding at least one surface of the cartridge to define an insertion direction and orientation for mating the cartridge with the opening. The embodiment of the receptor 200 further comprises a top portion 220 and a bottom portion 230. In one embodiment, the at least one photodetector is a spectrometer comprising linear diode array (each diode is referred to as a photodector), a reflecting grating and 256 pixels, and fiber optic connection to the spectrometer. Since the bend radius of fiber optic cables are limited, prisms are used to bend the EMR 90 degrees. As an example, the EMR is admitted through opening 267, and since there is sufficient space for bending the fiber optic cable, no prism is required. However, EMR emerging through opening 268 enters the spectrometer via a prism, in order to minimize the space between the receptor and the base of the analyzer.

Referring to FIG. 9J is illustrated a third perspective view of the receptor of a joint-diagnostic spectroscopic and biosensor analyzer shown in FIG. 9F, with the top portion of the receptor 220 and cartridge 10a hidden. The bottom portion 230 comprises a bed 237 for installing an optional heating element (not shown) for heating the biosensor conduit of the cartridge 10a, and a bed 239 for installing an optional heating element (not shown) for pre-heating the blood in the blood storage conduit 51a (see FIG. 8E). Also shown is a cavity 241 for housing a thermistor for regulating the heating elements. In this embodiment, the optional heaters are flexible heating elements having a plastic substrate and a layer of aluminum on the top. The aluminum functions as a heat spreader and makes the heater more durable for repeated insertion and removal of cartridges into the receptor. Attached to the substrate is pressure sensitive adhesive (PSA), for attaching the heater to the beds 237 and 239. In this embodiment, two heaters are connected and the thermistor for controlling the heaters is stuck to the PSA of the heater at bed 237. The thermistor (not shown), fits in the recess 241. Sufficient space is shown to the left of recess 241 for electrical connections.

Referring collectively to FIGS. 9F and 9J is shown a physical interface 219 attached to back portion 218 (see FIG. 9B) of receptor 200. Ports 233 are for facilitating electrical connection between biosensors 80a and physical interface 219, and ports 235 are for facilitating electrical connection between physical interface 219 and the analyzer processor. Although in this embodiment, the biosensors are configured to fit in the slot shown in 233, a person of ordinary skill in the art will appreciate that the analyzer input electrical contact can mate with the cartridge biosensor output electrical in different ways, for example, the input contact can be mechanically brought into contact with the top of the cartridge biosensor output electrical after the cartridge is in an operational position in the analyzer.

Referring to FIG. 9D is shown the top portion 220 of the receptor 200 comprises a spring-loaded locating element 201 for engaging with a notch 23 disposed at the top of the cartridge (see FIG. 8A) for forcing the cartridge against the heating elements.

Referring to FIGS. 9F-9J are illustrations of the bottom portion 230 of receptor 200, the cartridge 10a, and the association between the cartridge and receptor bottom portion 230. Illustrated in FIG. 9F is a second perspective view of a joint-diagnostic spectroscopic and biosensor cartridge 10a inserted in the receptor of an analyzer, with the top portion of the receptor hidden, and only showing the bottom portion 230. Illustrated in FIG. 9G is a perspective view of the cartridge 10a shown in FIG. 9F. Illustrated in FIG. 9H is a second perspective view of the cartridge 10a, showing the bottom cartridge housing member 30a.

Referring collectively to FIGS. 6A-8K, illustrated is an embodiment of a disposable cartridge 10a adapted for insertion along an insertion plane, substantially defined by the gasket 100a, into the receptor of a joint spectroscopic and biosensor analyzer for measurement of at least two hemoglobin species in a patient's blood sample by spectroscopy, and measurement of at least pH of the blood sample by biosensor, for assessing the patient's oxygenation and acid-base status. The gasket, may, for example, define an external surface of the cartridge that can cooperate with a surface of the receptor (or the opening of the receptor) to define the insertion plane. The cartridge comprises a housing having at least a first housing member 20a and a second housing member 30a bonded together by a gasket 100a. The housing comprises: a) a cartridge inlet 43a for receiving the blood sample; b) a blood storage conduit 51a (see FIG. 8E) having a proximal end close to the cartridge inlet and a distal end away from the cartridge inlet; c) an optical chamber 58a (see FIG. 8D) for receiving the blood from the distal end of the blood storage conduit and for measuring the at least two hemoglobin species, the optical chamber comprising an optical depth dimension orthogonal to the insertion plane; d) optical windows 67a and 38a positioned to align with at least a portion of the optical chamber 58a for collecting spectroscopic data from blood in that portion of the optical chamber; e) a biosensor conduit 54a (see FIG. 8D) for receiving the blood from the optical chamber overflow chamber, the biosensor conduit 54a having at least one biosensor for measuring the at least pH of the blood sample; f) a blood shunt 45a for providing fluid connectivity between the distal end of the blood storage conduit and the optical chamber overflow chamber, the blood shunt having a maximum shunt depth dimension orthogonal to the insertion plane, and wherein the maximum shunt depth dimension is substantially larger than the optical chamber depth dimension (see FIGS. 8D and 8J); g) an air bladder 85a and an air bladder exit port 87a within the housing (see FIGS. 8B, 8E and 8L) for providing pressurized air for urging blood from the blood storage conduit 51a into the biosensor conduit 54a; h) a waste receptacle 93a (see FIG. 8B) for receiving waste liquid from the biosensor conduit 54a; and i) a waste receptacle vent 95a (see FIG. 7B) for relieving pressure in the waste receptacle 93a.

Referring collectively to FIGS. 6B, 7D, 8D and 8J is illustrated the optical chamber overflow chamber comprising: a) a first duct shown as a hole 248 (see FIG. 6B) fluidly connected with the shunt 45a and traversing the thickness of the second housing member 30a; b) a recess 149 (see FIG. 7D) disposed at the bottom of the second housing member 30a and fluidly connected to the first duct 248; c) a second duct shown as a hole 148 (see FIG. 6B) having a first cross-sectional area along the cartridge insertion plane, and fluidly connected to the recess; and d) an enlarged cavity 56a having a second cross-sectional area parallel to the first cross-sectional area. The second cross-sectional area is substantially larger than the first cross-sectional area, whereby blood flow by capillary action slows down as the blood reaches the end of the second duct, and wherein the enlarged cavity is simultaneously in fluid connection with the optical chamber and the second duct.

Another embodiment of a disposable cartridge for operation with a joint spectroscopic and biosensor blood analyzer for measurement of at least two hemoglobin species in a patient's blood sample by spectroscopy, and measurement of at least pH of the blood sample by biosensor, for assessing the patient's oxygenation and acid-base status, comprises a housing having at least a first housing member and a second housing member bonded together by a gasket, wherein the housing comprises: a) a cartridge inlet; b) a blood storage conduit within the housing having a proximal end close to the cartridge inlet and a distal end away from the cartridge inlet; c) an optical chamber within the housing for receiving the blood from the distal end of the blood storage conduit and for measuring the at least two hemoglobin species; d) an optical chamber overflow chamber fluidly connected with the optical chamber; e) a biosensor conduit within the housing for receiving the blood from the optical chamber overflow chamber, the biosensor conduit comprising a proximal end, a distal end and at least a portion of a pH biosensor; f) a calibration fluid pouch for storing and releasing calibration fluid, a spike disposed in the second housing member of the cartridge for rupturing the pouch, a recess disposed in the opposite side of the second housing member, a hole in the spike for permitting flow of the calibration fluid from the pouch to the recess for channeling the calibration fluid to the biosensor conduit; g) a waste receptacle for receiving liquid waste from the biosensor conduit; h) a vent for relieving pressure in the waste receptacle; and i) an air bladder and an air bladder exit port within the housing for providing pressurized air for urging blood from the blood storage conduit into the biosensor conduit. The cartridge further comprises a compressible member surrounding the spike, for supporting the calibration fluid pouch.

Illustrated in FIG. 10 is a block diagram of an embodiment of a joint-diagnostic spectroscopic and biosensor analyzer. The embodiment comprises the following components: 1) cartridge receptor 200; 2) microprocessor 300; 3) source of electromagnetic radiation (EMR) 310; 4) spectrometer 320; 5) EMR source circuit board 330; 6) spectrometer circuit board 340; 7) biosensor circuit board 350; 8) limit switch 360 for notifying microprocessor that cartridge is fully inserted; 9) power supply 370; 10) heater controller 380; 11) stepper motor circuit board 390; 12) analyzer display screen 400; and 13) analyzer printer 410.

An embodiment of a joint spectroscopic and biosensor system for measurement of at least two hemoglobin species in a patient's blood sample by spectroscopy, and measurement of at least pH of the blood sample by biosensor, for assessing the patient's oxygenation and acid-base status comprises a disposable cartridge and an analyzer. The cartridge comprises a cartridge housing and the cartridge housing comprises: a) cartridge inlet; b) a blood storage conduit having a proximal end close to the cartridge inlet and a distal end away from the cartridge inlet; c) an optical chamber for receiving the blood from the distal end of the blood storage conduit and for measuring the at least two hemoglobin species, the optical chamber comprising a first optical window and a second optical window; d) an optical chamber overflow chamber fluidly connected with the optical chamber; e) a biosensor conduit for receiving the blood from the optical chamber overflow chamber, the biosensor conduit comprising a proximal end, a distal end and at least a portion of a pH biosensor; f) a pH biosensor electrical output element; g) a calibration fluid pouch containing calibration fluid for at least calibrating the pH biosensor; and h) an air bladder and an air bladder exit port.

The analyzer comprises an analyzer housing, the analyzer housing comprising: a) a receptor comprising a first opening for receiving and aligning the cartridge in an operational position; b) a source of electromagnetic radiation; c) at least one photodetector; d) a power supply; and e) a processor for controlling the analyzer. The receptor further comprises: i) a second opening for directing the electromagnetic radiation to the first optical window when the cartridge is in the operational position; ii) a third opening for directing electromagnetic radiation emerging from the second optical window to the at least one photodetector when the cartridge is in the operational position; iii) a physical interface for providing electrical contact between the pH biosensor electrical output element and the processor; and iv) a bracket mounted on the receptor for at least supporting a first stepper motor for applying force to the calibration fluid pouch against a spike for rupturing the spike and releasing calibration fluid, and a second stepper motor for forcing air from the air bladder through the air bladder exit port, for pushing the blood into the biosensor conduit.

In some embodiments, the receptor further comprises a top portion and a bottom portion, and the bottom portion comprises at least one heating element layered on the surface of the bottom portion, for heating the cartridge, and the top portion comprises a spring-loaded locating element for engaging with a notch disposed at the top of the cartridge, for forcing the cartridge against the at least one heating element.

The procedures for operating a joint spectroscopic and biosensor system comprising the analyzer illustrated in FIG. 10 and the cartridge illustrated collectively in FIGS. 1A-8K for measurement of at least two hemoglobin species in a patient's blood sample by spectroscopy, and measurement of at least pH of the blood sample by biosensor, for assessing the patient's oxygenation and acid-base status, comprises the following steps:
1. With cartridge placed on table (for blood in a syringe) or cartridge kept horizontal for capillary blood (via a Capillary Adaptor), fill blood storage conduit up to the Capillary Break (see 148 and 56a in FIG. 8J);
2. Use cap to make an air-tight seal with cartridge inlet (see FIG. 7B); and
3. Insert capped cartridge into receptor of analyzer (see FIG. 9A).
   The following steps are programmed in the microprocessor of the analyzer:
1. Optical reading begins when cartridge triggers limit switch 360 in FIG. 10 that indicates complete cartridge insertion;
2. Preferably following optical reading, since the time for optical reading is relatively short, stepper motor 215 ruptures the calibration fluid pouch 90a (see FIG. 9E), releasing calibration fluid into the biosensor conduit for wet-up of the biosensors and calibration of hydrated biosensors, facilitated by calibration fluid pouch depressor 205;
3. Following calibration of biosensors, with calibration fluid pouch depressor 205 in its last position, stepper motor 213 activates air bladder, facilitated by air bladder depressor 203, pushing the trailing end of blood sample, so that the blood displaces the calibration fluid and biosensor measurement of the blood is conducted.

An air bubble created in the enlarged cavity 56a and the conduit formed by connecting groove 57a positioned to provide fluid connection between enlarged cavity 56a and biosensor conduit groove 55a of cartridge 10a (see FIG. 6C) separates the blood and calibration fluid. The air bubble assists in purging the calibration fluid out of the biosensor conduit. With blood occupying the biosensor conduit (in place of calibration fluid), biosensor measurements are performed and analyzer informs user to remove and discard cartridge.

While the above description provides example embodiments, it will be appreciated that the present invention is susceptible to modification and change without departing from the fair meaning and scope of the accompanying claims. Accordingly, what has been described is merely illustrative of the application of aspects of embodiments of the invention. Numerous modifications and variations of the present invention are possible in light of the above teachings. It is therefore to be understood that within the scope of the appended claims, the invention may be practiced otherwise than as specifically described herein. Furthermore, the discussed combination of features might not be absolutely necessary for the inventive solution.

1. A disposable cartridge for operation with a joint spectroscopic and biosensor blood analyzer for measurement of at least two hemoglobin species in a patient's blood sample by spectroscopy, and measurement of at least pH of the blood sample by biosensor, for assessing the patient's oxygenation and acid-base status, the cartridge comprising:
a housing having at least a first housing member and a second housing member bonded together by a gasket, wherein the housing comprises
a cartridge inlet:
a blood storage conduit within the housing having a proximal end close to the cartridge inlet and a distal end away from the cartridge inlet;
an optical chamber within the housing for receiving the blood from the distal end of the blood storage conduit and for measuring the at least two hemoglobin species;
an optical chamber overflow chamber for receiving the blood from the optical chamber;
a biosensor conduit within the housing for receiving the blood from the optical chamber overflow chamber, the biosensor conduit comprising a proximal end, a distal end and at least a portion of a pH biosensor;
a waste receptacle for receiving liquid waste from the biosensor conduit;
a vent for relieving pressure in the waste receptacle;
an air bladder and an air bladder exit port within the housing for providing pressurized air for urging blood from the blood storage conduit into the biosensor conduit; and
an optical window and an aligned optical member, the first housing member comprising one of the optical window and the aligned optical member, and the second housing member comprising the other of the optical window and the aligned optical member; the aligned optical member being one of a reflecting member or a second optical window, and being positioned to align with at least a portion of the optical chamber and at least a portion of the optical window;
the gasket having
at least one gasket cut-out positioned to provide fluid connection between the blood storage conduit and the optical chamber, wherein at least a portion of the at least one gasket cut-out is positioned to align with at least a portion of the optical chamber for collecting spectroscopic data from blood in that portion of the optical chamber.

2. The disposable cartridge of according to claim 1, wherein the at least one gasket cut-out has a second portion positioned to align with the active area of the pH biosensor.

3. The disposable cartridge according to claim 1, wherein the disposable cartridge is insertable into a receptor of the joint spectroscopic and biosensor analyzer, and at least one of the first and second optical window is positioned to align with at least a portion of the optical chamber for collecting spectroscopic data from blood in that portion of the optical chamber, the housing further comprising:
a blood shunt for providing fluid connectivity between the distal end of the blood storage conduit and the optical chamber overflow chamber, wherein the optical chamber overflow chamber comprises:
a first duct fluidly connected with the blood shunt and traversing a thickness of the second housing member;
a recess disposed at the bottom of the second housing member and fluidly connected to the first duct;
a second duct having a first cross-sectional area, and fluidly connected to the recess; and
an enlarged cavity having a second cross-sectional area parallel to the first cross-sectional area; wherein, the second cross-sectional area is substantially larger than the first cross-sectional area, whereby blood flow by capillary action slows down as the blood reaches the end of the second duct, and wherein the enlarged cavity is simultaneously in fluid connection with the optical chamber and the second duct.

4. The disposable cartridge of claim 3, wherein the disposable cartridge is insertable along a plane substantially defined by a surface of the gasket, into the receptor of the joint spectroscopic and biosensor analyzer, the optical chamber comprising an optical depth dimension orthogonal to the plane, the blood shunt having a maximum shunt depth dimension orthogonal to the plane, the maximum shunt depth dimension being substantially larger than the optical chamber depth dimension, and the first cross-sectional area being along the plane.

5. The disposable cartridge of claim 1, wherein the optical chamber overflow chamber is fluidly connected with the optical chamber, and the housing further comprises:
a calibration fluid pouch for storing and releasing calibration fluid;
a spike disposed in the second housing member of the cartridge for rupturing the calibration fluid pouch;
a recess disposed in the opposite side of the second housing member; and
a hole in the spike for permitting flow of the calibration fluid from the calibration fluid pouch to the recess for channeling the calibration fluid to the biosensor conduit.

6. The disposable cartridge according to claim 5, wherein the cartridge further comprises a compressible member surrounding the spike, for supporting the calibration fluid pouch.

7. A system for transferring capillary blood from a puncture site of a body part of a patient, the system comprising:
a capillary adaptor; and
the disposable cartridge according to claim 1, wherein the cartridge inlet engages the capillary adaptor,
the cartridge inlet further comprising:
an internal wall for receiving the capillary adaptor, the internal wall defining an airflow path for airflow between an exterior of the disposable cartridge and the blood storage conduit when the capillary adaptor is being removed from the cartridge inlet:
an external wall having a cartridge inlet thread;
a blood storage conduit entrance at the base of the cartridge inlet, the blood storage conduit beginning at the blood storage conduit entrance; and
a cartridge inlet inner face surrounding the blood storage conduit entrance;
the capillary adaptor comprising:
a capillary adaptor inlet member comprising a capillary adaptor tube, the capillary adaptor inlet member having a capillary adaptor inlet port for receiving the blood sample;
a capillary adaptor outlet member sized to fit into the cartridge inlet;
a capillary adaptor outlet port disposed at the end of the capillary adaptor outlet member;
a capillary adaptor face surrounding the capillary adaptor outlet port;
a capillary adaptor lumen extending from the capillary adaptor inlet port to the capillary adaptor outlet port;
a handgrip for handling the capillary adaptor; and
an internal wall in the hand grip having a capillary adaptor thread for engaging the cartridge inlet thread in the cartridge inlet;
wherein when the capillary adaptor thread is properly engaged with the cartridge inlet thread, the capillary adaptor face mates with the cartridge inlet inner face, sufficiently to permit flow of blood from the patient to the blood storage conduit by capillary action.

8. The system according to claim 7, wherein the position of the capillary adaptor face relative to the cartridge inlet inner face, when the capillary adaptor is fully engaged with the cartridge inlet, is one of no gap between the capillary adaptor face.

9. The system according to claim 7, wherein the cartridge inlet thread is an abbreviated thread.

10. The system according to claim 7, wherein the capillary adaptor thread is an abbreviated thread.

11. The system according to claim 7, wherein the volume of the capillary adaptor lumen is in the approximate range of about 5 microliters to about 20 microliters.

12. The system according to claim 7, wherein the volume of the capillary adaptor lumen is in the approximate range of about 5 microliters to about 10 microliters.

13. The system according to claim 7, wherein the length of the capillary adaptor inlet member is in the approximate range of about 2 millimeters to about 5 millimeters.

14. A system for transferring blood from a syringe containing the blood, the system comprising:
the disposable cartridge according to claim 1, wherein the cartridge inlet engages the syringe,
the cartridge inlet further comprising:
an internal wall for receiving the syringe, the internal wall defining an airflow path for airflow between an exterior of the disposable cartridge and the blood storage conduit when the syringe is being removed from the cartridge inlet;
an external wall;
a blood storage conduit entrance at the base of the cartridge Inlet, the blood storage conduit beginning at the blood storage conduit entrance; and
a cartridge inlet inner face surrounding the blood storage conduit entrance.

15. A joint spectroscopic and biosensor system for measurement of at least two hemoglobin species in a patient's blood sample by spectroscopy, and measurement of at least pH of the blood sample by biosensor, for assessing the patient's oxygenation and acid-base status, the system comprising:
the disposable cartridge according to claim 1, wherein
the first optical window and the second optical window are part of the optical chamber;
the optical chamber overflow chamber is fluidly connected with the optical chamber, and
the housing further comprises:
a pH biosensor electrical output element; and
a calibration fluid pouch containing calibration fluid for at least calibrating the pH biosensor; and
an analyzer comprising an analyzer housing, wherein the analyzer housing comprises:
a receptor comprising a first opening for receiving and aligning the cartridge in an operational position;
a source of electromagnetic radiation;
at least one photodetector;
a power supply; and
a processor for controlling the analyzer; and
wherein
the receptor further comprises:
a second opening for directing the electromagnetic radiation to the first optical window when the cartridge is in the operational position;
a third opening for directing electromagnetic radiation emerging from the second optical window to the at least one photodetector when the cartridge is in the operational position;
a physical interface for providing electrical contact between the pH biosensor electrical output element and the processor; and
a bracket mounted on the receptor for at least supporting a first stepper motor for applying force to the calibration fluid pouch against a spike for rupturing the calibration fluid pouch to release the calibration fluid, and a second stepper motor for forcing air from the air bladder through the air bladder exit port, for pushing the blood into the biosensor conduit.

16. The system according to claim 15, wherein the receptor further comprises a top portion and a bottom portion, and the bottom portion comprises at least one heating element layered on a surface of the bottom portion, for heating the cartridge, and the top portion comprises a spring-loaded locating element for engaging with a notch disposed at a top of the cartridge, for forcing the cartridge against the at least one heating element.

17. The system as defined in claim 7 further comprising a cap for covering the cartridge inlet when one of the capillary adaptor and the syringe is withdrawn from the cartridge inlet, wherein the cap comprises a cap airflow path for airflow between an exterior of the disposable cartridge and the blood storage conduit when the cap is being engaged to impede blood in the blood storage conduit being disturbed by compression of air within the cartridge inlet during engagement of the cap.
